SEQUENCE LISTING (8 sequences)

SEQ ID NO: 1 - DNA, 19 bp, Artificial sequence
  Amplification primer
cgaggccctgtaattggaa

SEQ ID NO: 2 - DNA, 17 bp, Artificial sequence
  Amplification primer
cggctgctggcaccaga

SEQ ID NO: 3 - DNA, 22 bp, Artificial sequence
  Probe
cgaggatccattggagggcaag

SEQ ID NO: 4 - DNA, 64 bp, Artificial sequence
  Template sequence
cttgccctccaatggatcctcgttaaaggatttaaagtggactcattccaattacagggcctcg

SEQ ID NO: 5 - DNA, 28 bp, Artificial sequence
  Probe
ggaatgagtccactttaaatcctttaac

SEQ ID NO: 6 - DNA, 28 bp, Artificial sequence
  Probe
tggaatgagtccactttaaatcctttaa

SEQ ID NO: 7 - DNA, 28 bp, Artificial sequence
  Probe
ttggaatgagtccactttaaatccttta

SEQ ID NO: 8 - DNA, 28 bp, Artificial sequence
  Probe
attggaatgagtccactttaaatccttt

(19) United States
(12) Patent Application Publication
(10) Pub. No.: US 2005/0026166 A1
(43) Pub. Date: Feb. 3, 2005

(54) Concurrent enzymatic polynucleotide synthesis and detectable signal generation

(76) Inventor: Wanli Bi, San Ramon, CA (US)

Correspondence Address: GRAY CARY WARE & FREIDENRICH LLP, 153 TOWNSEND, SUITE 800, SAN FRANCISCO, CA, 94107, US

(21) Appl. No.: 10/731,214

(22) Filed: Dec. 8, 2003

Related U.S. Application Data
(60) Provisional application No. 60/431,822, filed on Dec. 9, 2002.

(51) Int. Cl. C12Q 1/68; C07H 21/04

(52) U.S. Cl. 435/6; 536/25.32

(57) ABSTRACT

The present invention is based, in part, on the discovery that structure specific nuclease, alone or in combination with nucleic acid polymerase, especially the ones lacking 5′ nuclease activity can be used for detection or measurement of target nucleic acid sequences. Accordingly the present invention provides methods and kits useful for generating signals indicative of the present of target nucleic acid sequences and for detection and/or measurement of target nucleic acid sequences.

This application claims priority under 35 U.S.C. §119(e) from provisional application No. 60/431,822 filed on Dec. 9, 2002.

### FIELD OF THE INVENTION

The present invention is related to generating and detecting nucleic acid sequences.

### BACKGROUND OF THE INVENTION

Nucleic acid amplification technologies are widely used in clinical microbiology, blood screening, food safety, genetic disease diagnosis and prognosis, environmental microbiology, drug target discovery and validation, forensics, and other biomedical research. It is becoming an essential part of emerging pharmacogenomics, prenatal diagnoses, and molecular based cancer diagnoses and therapy. Consequently detection of amplified product becomes ever so critical especially being able to determine robustness of nucleic acid testing, specificity, sensitivity, reliability in terms of accuracy and precision, and affordability.

Nucleic acid sequence specific amplification allows sensitive detection of the presence of a specific sequence. A major breakthrough in nucleic acid amplification was the invention of polymerase chain reaction (PCR). Following the invention of PCR, many other amplification methods, either thermocyling or isothermal amplification methods, have been developed. Analysis of amplification and detection of amplified products are important aspects for each method.

Detection, especially detection of amplified products should confer the following features.

(a) High specificity. A sequence specific probe can be used to accomplish this. To minimize any possible side reaction and consequent false positive result, the probe usually should not be involved in amplification.

(b) No interference with amplification. Detection of amplified products should have little or no effect on the amplification process. It should not reduce specificity or lower sensitivity either.

(c) Simplicity. Detection methods should have minimal post-amplification manipulation, ease of use, high throughput and affordability, which are all or at least partially dependent on simplicity.

(d) Concurrence of amplification and detection. This allows one to monitor amplification in real-time, which allows accurate quantification with a broad dynamic range. Target quantification is important in various applications, e.g., measurement of viral load.

(e) A closed homogeneous system. Such a system can greatly reduce cross contamination by product carryover. Billion-fold amplification is routinely achieved with various methods. Any post-amplification manipulation of amplified products posts a serious risk of cross contamination. To minimize the risk, amplification and detection should be conducted in a closed system.

Isothermal amplification refers to a category of amplification in which amplification is carried out at a substantially constant temperature. Transcription-mediated amplification (TMA), nucleic acid sequence based amplification (NASBA), strand-displacement amplifcation (SDA), rolling circle amplification (RCA), single primer isothermal amplification (SPIA), and exponential single primer isothermal amplification (X-SPIA™) (disclosed in U.S. Pat. Nos. 5,130,238; 5,409,818; 5,554,517; 6,063,603; 5,399,491; 5,437,990; 5,480,784; 5,888,779; 6,090,591; 5,270,184; 5,916,779; 5,854,033; 6,183,960; 6,210,884; 6,344,329, 6,251,639), self-sustained sequence replication (3SR) (Guatelli, 1990) and loop mediated isothermal amplification (LAMP) (Notomi, 2000; U.S. Pat. No. 6,410,278) are examples of isothermal amplification. Various homogeneous real-time detection means, including fluorescence polarization (FP) (Walker, 1996; Spears, 1997), restriction endonuclease-mediated cleavage of a FRET probe (Nadeau, 1999), molecular beacon (Leone, 1998; Nilsson, 2002), have been explored.

FP and cleavage of a FRET probe were used in detection of SDA products. The main drawback for both methods is that they both require the probe to participate in amplification. A probe being extendable by DNA polymerase makes it available for side reactions including non-specific reactions and reactions between oligonucleotides. Such side reactions in turn can lead to false positive results. A more specific detection system of SDA products remains to be developed.

Leone and Nilsson reported uses of molecular beacon in detection of amplified products in NASBA and RCA respectively. Although molecular beacon probe in either amplification scheme does not participate amplification process, its use is problematic. For RCA amplification, molecular beacon application is limited to linear amplification scheme. In exponential form of RCA, any hybridized molecular beacon molecules will be displaced, thus it is impossible to generate stable detection signals. Furthermore, linear RCA is not sensitive enough for most applications. Even with 5×10⁸ target molecules, an impractically high level, detection signal generated by RCA—molecular beacon was merely marginally distinguishable from negative control (Nilsson, 2002).

The application of molecular beacon in detection of NASBA products is also problematic. Leone used a molecular beacon to detect amplified products. The molecular beacon was designed to bind to RNA transcripts. Binding of the molecular beacon, a DNA oligonucleotide, to RNA transcripts makes the RNA prone to RNase H degradation. Both RNA and RNase H are two essential components for NASBA. Any RNA degradation will interrupt amplification because intactness of the RNA transcript is essential to amplification by NASBA.

In addition, application of molecular beacon faces the dilemma of signal generation versus template amplification. While signal generation requires stable hybridization of a probe to a template, such stable hybridization can stall amplification. Whereas efficient template amplification requires any hybridized probe molecules being displaced, such probe displacement results in a low or no detectable signal: Therefore, quantification of target molecule is not very accurate and robust.

TMA and 3SR are isothermal amplification methods similar to NASBA. They face the same issues associated with using molecular beacon in NASBA amplification methods. It is necessary to find a detection means to generate stable and intense signal and resolve the conflict between signal generation and template amplification.

The only homogeneous closed detection was used in LAMP and was based on intercalator ethidium bromide (Nagamine, 2002). Detection by intercalator is not specific. In an amplification system like LAMP in which four primers are required for amplification, eliminating non-specific reaction is a great challenge.

Therefore there is a need in the art to develop a probe based sequence specific homogeneous detection system for isothermal amplifications.

Thermacycling amplification methods such as PCR utilize two oligonucleotides, DNA polymerization components and a thermocycling machine to copy a specific sequence exponentially. Several closed homogeneous assay systems have been developed to detect amplified products generated by thermacycling amplification. One way is to use a nucleic acid intercalator which favorably binds to double-stranded DNA and yields higher fluorescence (U.S. Pat. Nos. 5,994,056 and 6,171,785).

Although it provides a homogeneous system to detect amplification products, the disadvantage is that it detects amplification in a non-sequence specific manner. Intercalator binds to any double strand sequence and produces higher fluorescence. Not only does it detect double stranded amplified sequence of interest but also double stranded side products. In the absence of target of interest and presence of side products, it yields false positive results. For the same reason, amplification and detection by this approach can produce wrong results especially when level of target sequence is low.

Recently a 5′ nuclease assay has been developed and is disclosed in U.S. Pat. Nos. 5,210,015; 5,487,972; 5,804,375 and 6,214,979. In the 5′ nuclease assay, a third oligonucleotide, most common a fluorescense resonance energy transfer (FRET) probe containing a fluorophore and a quencher group, is included in PCR amplification reaction. Upon binding to target sequence, the probe is degraded by 5′ nuclease activity of certain DNA polymerases such as Taq and Tth DNA polymerase. Physical separation of fluorophore and quencher results in increased fluorescence. By monitoring change in fluorescence, presence of target sequence is detected qualitatively and quantitatively.

The problem with 5′ nuclease assay is that it cannot be used in PCR with high fidelity DNA polymerases. High fidelity DNA polymerases refer to those DNA polymerases with 3′ to 5′ exonuclease or proofreading activity. Those enzymes generally do not have required 5′ nuclease activity. For many applications, for instance, clinical microbiology and blood screening, high fidelity PCR amplification is of great importance. It is desired and critical to use DNA polymerase with highest fidelity to effectively minimize mutations introduced during thermocycling amplification. If such mutations occur in probe binding region, they can produce qualitatively and/or quantitatively wrongful results.

Taq DNA polymerase, the most widely used DNA polymerase in 5′ nuclease assay, along with other DNA polymerases having 5′ nuclease activity do not have high fidelity in comparison with other DNA polymerases. As a matter of fact, Pfu, Tli, and KlenTaq DNA polymerase have 8.0-, 3.7- and 2.4-fold lower mutation rate than Taq DNA polymerase respectively (Lundberg, 1991; Cariello, 1991; and Barnes, 1992). However these DNA polymerases either have proof-reading activity (Pfu and Tli DNA polymerases) or no nuclease activity at all (KlenTaq). Neither of them is suitable for 5′ nuclease assay because of lack of 5′ nuclease activity. When target abundance is low, it takes more cycles to get enough amplified product to reach detectable level. The more cycles the amplification undergoes, the more mutations are created. High fidelity enzyme is needed even more in such situation. In addition to having higher fidelity, those enzymes have been demonstrated higher thermostability than that of Taq DNA polymerase. Higher thermostability means higher amplification yield and higher signal to noise ratio.

Currently there is a need to use DNA polymerases with higher fidelity and thermostability to amplify target sequence more faithfully with higher yield.

U.S. Pat. No. 5,925,917 discloses use of molecular beacon to detect amplification. A FRET probe comprising a stem-loop formed by 5′ and 3′ ends is employed in the approach. A fluorophore and a quencher group are attached to the 5′ and 3′ end respectively. In non-hybridized form, fluorescence of the fluorophore is greatly quenched due to its spatial proximity to the quencher. Hybridization to target sequence stretches distance between the fluorophore and disrupts energy transfer between the two groups. Consequently there is an increase of fluorescence which indicates the presence of target sequences. Although molecular beacon provides a way to detect amplification with enzymes having no 5′ nuclease activity, there are problems associated with this approach.

One problem associated with molecular beacon is that the fluorescence increase is significantly lower than the fluorescence increase by degradation of the probe in the 5′ nuclease assay. Hybridization could only partially release the quenching effect while cleavage fully releases fluorescence quenching.

Another problem associated with molecular beacon is that hybridized probe can be inhibitory to amplification. Without being cleaved, the hybridized fluorogenic probe may remain hybridized and block further amplification. It was reported that a hybridized non-extendable oligonucleotide inhibited PCR amplification with a 5′ nuclease deficient DNA polymerase (Yu, 1997). A hybridized molecular beacon molecule, which is not extendable, should behave very much like the oligonucleotide used in Yu's work and inhibit PCR amplification with a 5′ nuclease deficient DNA polymerase. So far most works, if not all, on using molecular beacon for PCR product detection, used intact Taq DNA polymerase (Tyagi, 1996; Piatek, 1998; Tyagi, 1998; Vet, 1998; Tyagi, 2000). There has been no published data showing that molecular beacon could be used in homogeneous PCR product detection with a 5′ exo/endonuclease deficient DNA polymerase.

In addition, displacement of hybridized probe means no fluorescence increase. All these problems make efficient amplification and product detection very difficult in using molecular beacon and a 5′ nuclease deficient DNA polymerase.

In addition to the above target amplification methods, there has also development in means to detect the presence of target nucleic acid via signal amplification. Because there is no target sequence amplification, signal amplification completely avoids carryover of amplified target sequence. Two nucleic acid cleavage based signal amplifications are cycling probe (U.S. Pat. Nos. 5,403,711; 5,011,769) and invader assay (U.S. Pat. Nos. 6,348,314; 6,090,543; 6,001,567; 5,985,557; 5,846,717; 5,837,450). None of them involve target sequence amplification or modification. Detectable signal is generated by enzymatic cleavage of hybridized probe. The cleavage also thermodynamically promotes removal of cleaved probe from target sequence. Probe undergoes a cycle of hybridization and cleavage in the presence of target sequence. Signal intensity is linearly proportional to the amount of target sequence presented in a sample.

In cycling probe assay, RNase H enzyme and a ribonucleotide containing probe are used for DNA sequence detection. In invader assay the amplification system contains a flap endonuclease and two partially overlapping oligonucleotides which form an invasive structure upon binding to target sequence and trigger probe cleavage. For example, each target sequence is estimated to evoke up to 3,000 cleavage events in invader assay (Lyamichev, 1999). Signal amplification can be increased 10⁷ cleavage events per target sequence by performing two consecutive invader assays (Hall, 2000). Nevertheless, it is still far lower than exponential amplification, e.g. PCR. Serial invader assay usually has higher background than regular invader assay, which has limited its application. Preamplification of target sequence by PCR is a common practice in invader assay (Olivier, 2002; Hsu, 2001; Mein, 2000) even though it is time consuming, costly, inconvenient and posts risk of product cross contamination.

U.S. Pat. No. 6,350,580 discloses a method to detect a target nucleic acid using a probe containing a secondary structure and a FEN nuclease. When target molecules are present, such probe binds to them and gets cleaved by FEN nuclease. Otherwise such probe exists in a conformation of its secondary structure. The secondary structure of the probe is a key component for the method, which causes many disadvantages.

First, it is difficult to design a probe with a suitable secondary structure. Such probe requires two melting temperatures (Tms), one for the secondary structure and the other for target hybridization. In the presence of target sequence, probe may stay in two forms, either in a form of secondary structure or in a form of hybridization. The two forms inevitably compete with each other. Consequently Tm of hybridization will be affected by Tm of secondary structure. Therefore the requirement of secondary structure(s) makes it very difficult to predict Tm of hybridization, which is critical for designing probes for target detection.

Second, it is very difficult to prepare a probe with a suitable secondary structure. Due to less than 100% coupling efficiency of solid phase oligonucleotide synthesis, oligonucleotide/probe chemically synthesized is a mixture of right length and shortened ones. The longer a probe is, the more the shortened ones in the final probe mixture. Addition of secondary structure forming sequence makes a probe longer than a probe without such a sequence, which reduces the yield for such probe and increases the cost.

Third, purification of oligonucleotides with secondary structure is difficult. Post-synthesis probe purification is a very important step in probe production. It is a well-known fact that the presence of stable secondary structure in an oligonucleotide can complicate oligonucleotide purification in various commonly used purification methods, e.g., polyacriamide gel electrophoresis (PAGE), anion exchange high performance liquid chromatography (AEX-HPLC), and reverse phase high performance liquid chromatography (RP-HPLC) (Wu, 1984; McLaughlin, 1984; Applied Biosystems, 1994). Probes are usually purified by one or more of these three methods. Multiple bands on PAGE, multiple peaks and unusual retention time on AEX-, RP-HPLC were observed with secondary structure containing oligonucleotides.

In addition, non-specific cleavage of secondary structure containing probe by FEN nuclease makes any detection based on this approach less reliable. There are many reports on cleavage of secondary structures by FEN. Recessed 5′ end (Harrington, 1994; Matsui, 1999; Rumbaugh, 1999; Bae, 1999), nicked duplex (Murante, 1995; Matsui, 1999; Hosfield, 1998), Gapped duplex (Wu, 1996; Matsui, 1999), pseudo-Y structure (Henricksen, 2000; Kaiser, 1999; Hosfield, 1998; Rao, 1998; Bae, 1999), Hairpin substrate (Kaiser, 1999) are cleaved by various FEN nucleases. Therefore detection by this method will produce high background which may severely limit detection sensitivity or yield false positive results.

In summary, there is a need in the art to develop more methods and kits useful for target nucleic acid detection and measurement.

### SUMMARY OF THE INVENTION

The present invention is based, in part, on the discovery that structure specific nuclease, alone or in combination with nucleic acid polymerase, especially the ones lacking 5′ nuclease activity can be used for detection or measurement of target nucleic acid sequences. Accordingly the present invention provides methods and kits useful for generating signals indicative of the present of target nucleic acid sequences and for detection and/or measurement of target nucleic acid sequences.

In one embodiment, the present invention provides a method for generating a signal indicative of the presence of a target nucleic acid sequence in a sample. The method comprises incubating a sample, a probe, and a structure specific nuclease, wherein the probe has a signaling moiety, is capable of hybridizing to a target nucleic acid sequence, and does not have a secondary structure that changes upon hybridizing of the probe to the target nucleic acid sequence, wherein the signaling moiety is inactivated when the probe is not hybridized to the target nucleic acid sequence, wherein the signaling moiety generates a signal by the structure specific nuclease when the probe is hybridized to the target nucleic acid sequence to form a suitable substrate for the structure specific nuclease, and wherein the generation of the signal is indicative of the presence of a target nucleic acid sequence in the sample.

In another embodiment, the present invention provides a method for detecting or measuring a target nucleic acid sequence in a sample. The method comprises incubating a sample, a probe, and a structure specific nuclease, wherein the probe has a signaling moiety, is capable of hybridizing to a target nucleic acid sequence, and does not have a secondary structure that changes upon hybridizing of the probe to the target nucleic acid sequence, wherein the signaling moiety is inactivated when the probe is not hybridized to the target nucleic acid sequence and the signaling moiety is activated to generate a signal by the structure specific nuclease when the probe is hybridized to the target nucleic acid sequence to form a suitable substrate for the structure specific nuclease, detecting or measuring the amount of the signal, which is indicative of the amount of the target nucleic acid sequence present in the sample.

In yet another embodiment, the present invention provides a method for generating a signal indicative of the presence of a target nucleic acid sequence in a sample. The method comprises incubating a sample, a first probe, and a structure specific nuclease and amplifying a sequence containing a target nucleic acid sequence in the sample including using a nucleic acid polymerase which does not have any substantial 5′-3′ nuclease activity wherein the first probe has a signaling moiety and is capable of hybridizing to the target nucleic acid, wherein the signaling moiety is inactivated when the first probe is not hybridized to the target nucleic acid sequence, wherein the signaling moiety generates a signal by the structure specific nuclease when the first probe is hybridized to the target nucleic acid sequence to form a suitable substrate for the structure specific nuclease, and wherein the generation of the signal is indicative of the presence of a target nucleic acid sequence in the sample.

In still another embodiment, the present invention provides a method for detecting or measuring the presence of a target nucleic acid sequence in a sample. The method comprises incubating a sample, a first probe, and a structure specific nuclease, amplifying a sequence containing a target nucleic acid sequence in the sample including using a nucleic acid polymerase which does not have any substantial 5′-3′ nuclease activity wherein the first probe has a signaling moiety and is capable of hybridizing to the target nucleic acid, wherein the signaling moiety is inactivated when the first probe is not hybridized to the target nucleic acid sequence, and wherein the signaling moiety generates a signal by the structure specific nuclease when the first probe is hybridized to the target nucleic acid sequence to form a suitable substrate for the structure specific nuclease, and detecting or measuring the amount of signal, which is indicative of the amount of the target nucleic acid sequence present in the sample.

In another embodiment, the present invention provides a kit which comprises a first probe and a structure specific nuclease, wherein the first probe has a signaling moiety, is capable of hybridizing to a target nucleic acid sequence, and does not have a secondary structure that changes upon hybridizing of the first probe to the target nucleic acid sequence, and wherein the signaling moiety is inactivated when the first probe is not hybridized to the target nucleic acid sequence and the signaling moiety is activated to generate a signal by the structure specific nuclease when the first probe is hybridized to the target nucleic acid sequence to form a suitable substrate for the structure specific nuclease.

In yet another embodiment, the present invention provides a kit which comprises a first probe, a structure specific nuclease, and a nucleic acid polymerase which does not have any substantial 5′-3′ nuclease activity, wherein the first probe has a signaling moiety and is capable of hybridizing to the target nucleic acid sequence, wherein the nucleic acid polymerase is capable of amplifying a sequence containing the target nucleic acid sequence, wherein the signaling moiety is inactivated when the first probe is not hybridized to the target nucleic acid sequence, and wherein the signaling moiety generates a signal by the structure specific nuclease when the first probe is hybridized to the target nucleic acid sequence to form a suitable substrate for the structure specific nuclease.

### BRIEF DESCRIPTION OF DRAWINGS

FIGS. 1A-1J illustrate structures which can be recognized and cleaved by a structure specific nuclease.

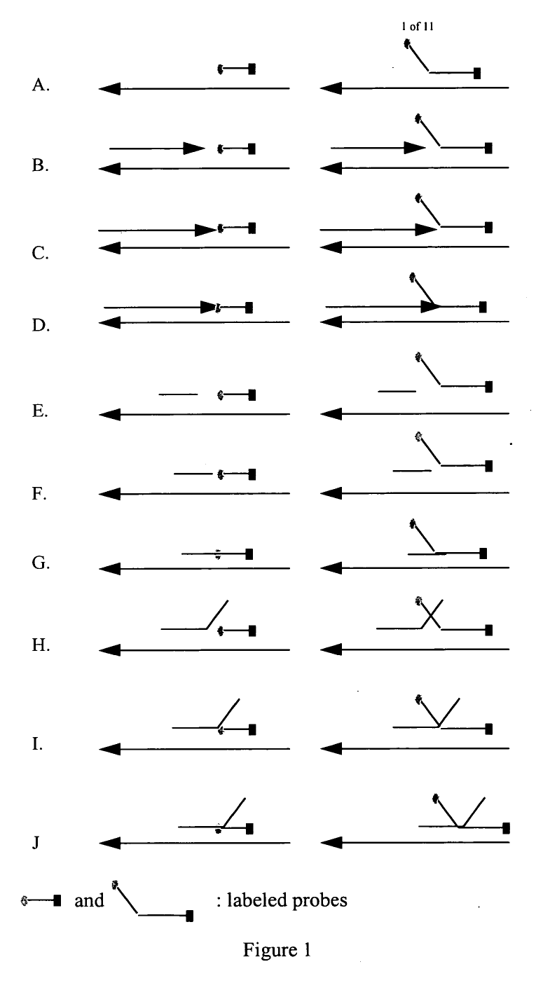

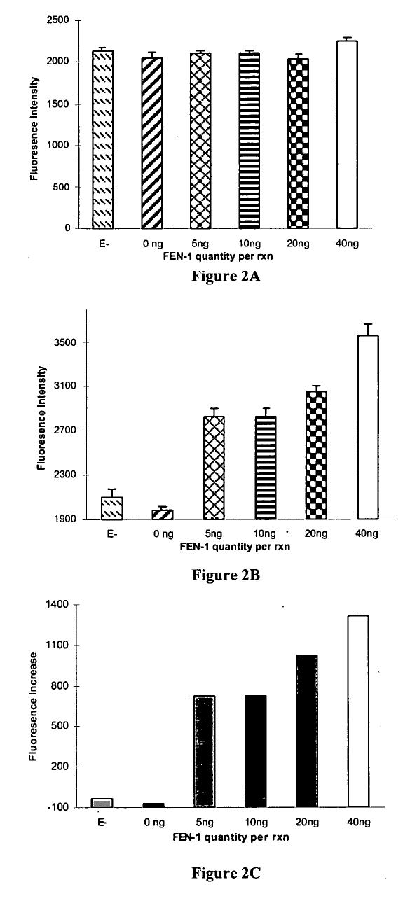

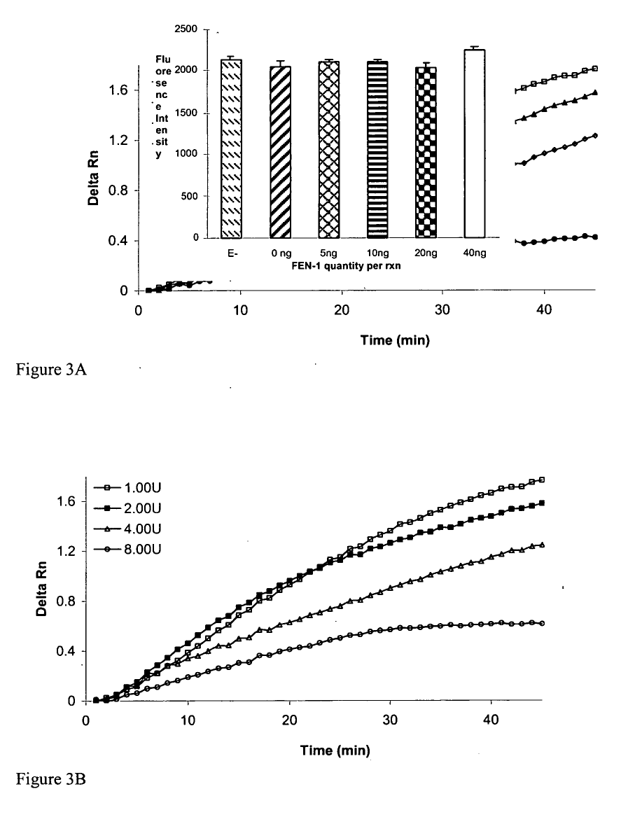

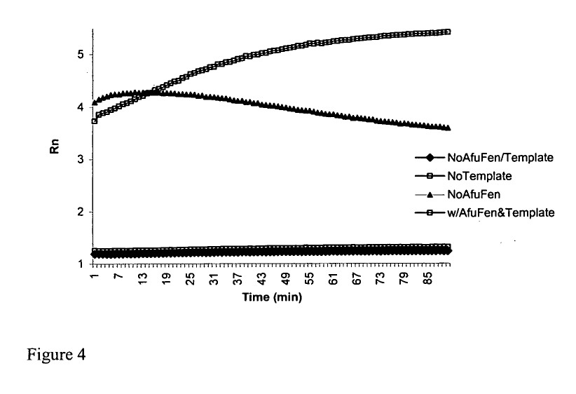

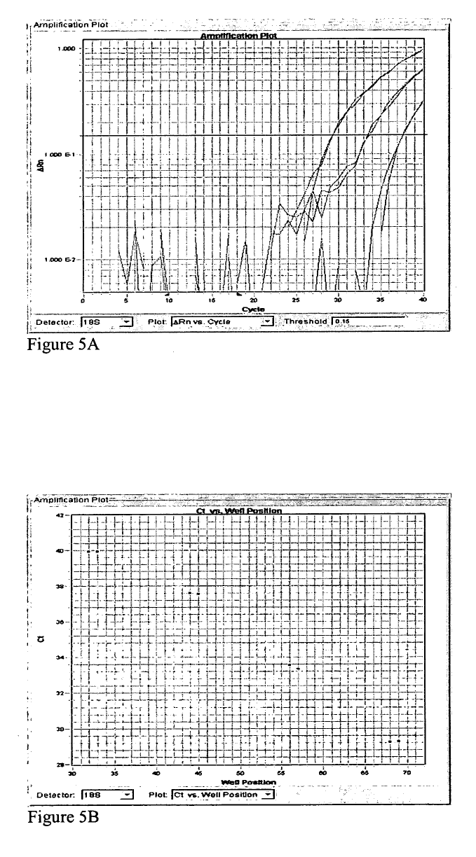

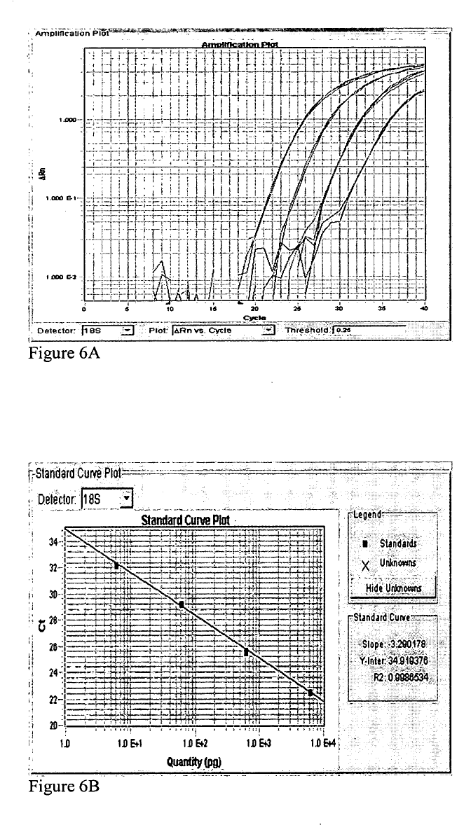

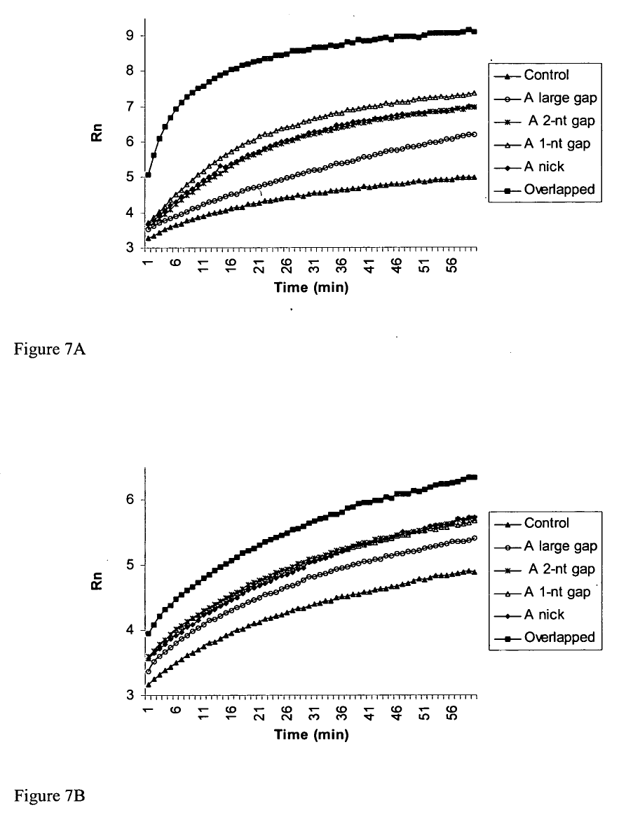

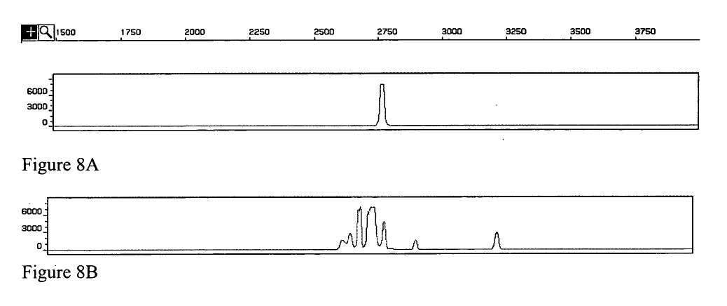

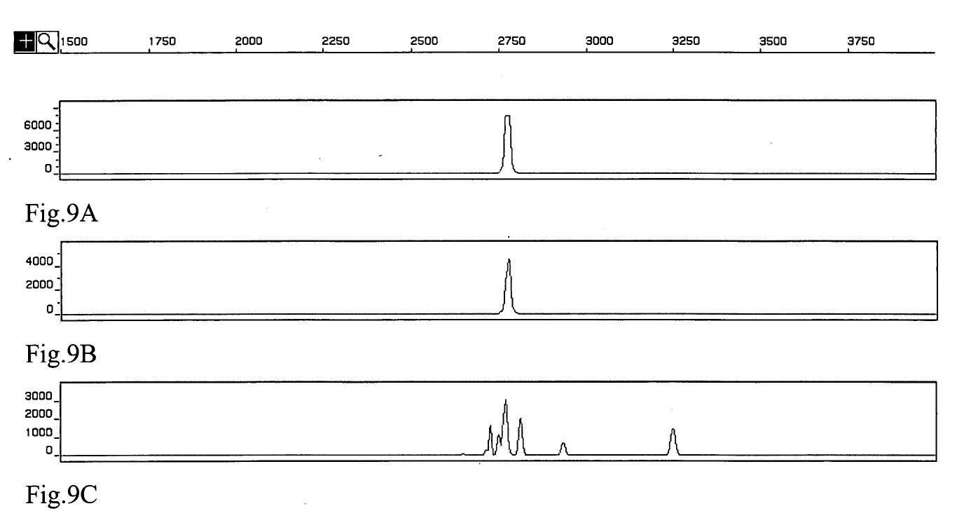

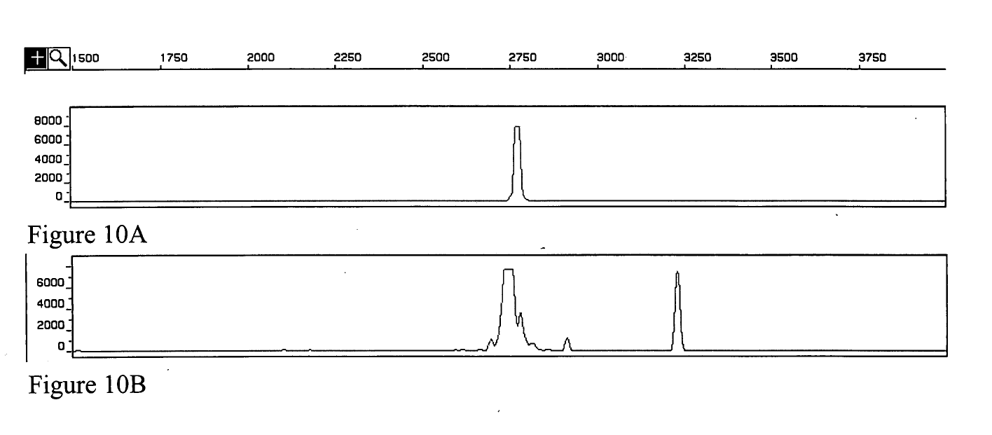

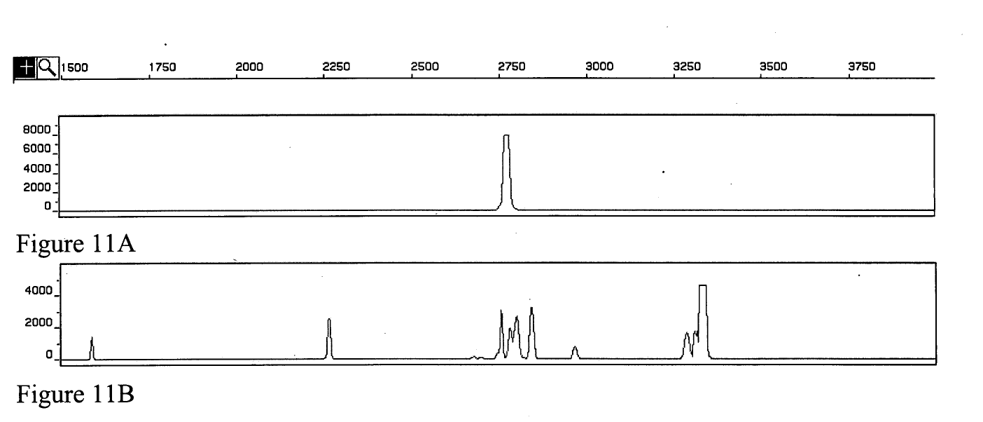

Left panel shows cleavage structures formed with a probe without a 5′ flap. Right panel shows cleavage structures formed with a probe with a 5′ flap. Bi-molecular cleavage structures comprising a template molecule and a probe molecule are shown in FIG. 1A. Tri-molecular cleavage structure is formed with a template molecule, a probe, and a third oligonucleotide (FIGS. 1B-1J). When hybridized to the template, there may be a gap, a nick or an overlapping sequence between the probe and the third oligonucleotide. Gapped, nicked and overlapped structures are shown in FIGS. 1B, 1E, and 1H, FIGS. 1C, 1F, and 1I, and FIGS. 1D, 1G, and 1J respectively. FIGS. 1B-1D are cleavage structures formed with an oligonucleotide which participates amplification or its extended form, along with a probe and a template molecule. FIGS. 1E-1J depict cleavage structures with a non-extendable oligonucleotide not involved in amplification, a probe, and a template molecule. The non-extendable oligonucleotide may either have a 3′flap (FIGS. 1H-1J) or have no 3′ flap (FIGS. 1E-1G).

FIGS. 2A-2C show nucleic acid detection with a structure specific nuclease and a DNA polymerase with strong strand displacement activity.

DNA polymerase from Bacillus stearothermophilus has strong strand displacement activity. A deletion mutant, Bst DNA polymerase LF which has no 5′ endo/exonuclease domain, has been widely used in SDA, RCA, LAMP, SPIA™ and X-SPIA™ processes. The structure specific nuclease used is flap endonuclease-1 from Archaeglobus fulgidus (Afu FEN-1). A FRET probe contains a fluorophore 6Fam at its 5′ and a black hole quencher-1 (BHQ-1) at its 3′ end. When the probe is intact, fluorescence level is low. Primer extension by Bst DNA polymerase LF creates a cleavage structure which is recognized by Afu FEN-1. Cleavage of the FRET probe within the cleavage structure physically separates 6Fam and BHQ-1. Consequently there is an increase in 6Fam fluorescence. This cleavage is template dependent as demonstrated in FIG. 2A. In the absence of template, no fluorescence increase was observed even with 40 ng Afu FEN-1. FIG. 2B shows fluorescence change with different amount of Afu FEN-1 in the presence of template molecules. Subtraction of fluorescence of no template control reactions (FIG. 2A) from FIG. 2B reflects net increase of fluorescence. The results are shown in FIG. 2C.

FIGS. 3A-3B show real-time detection of strand elongation by Bst DNA polymerase LF.

FIG. 4 shows real-time detection of DNA synthesis by reverse transcriptase.

FIGS. 5A-5B show PCR amplification of human genomic DNA with a high fidelity DNA polymerase.

Pfu DNA polymerase was isolated from a hyperthermophilic archaebacteria. It has proof-reading activity and no 5′ nuclease activity. Its fidelity is 8-fold higher than Taq DNA polymerase, the most commonly used DNA polymerase in PCR. Its thermostability, a key feature for an enzyme used in thermocycling amplification, is much higher than that of Taq DNA polymerase. Inclusion of a structure specific nuclease, Afu FEN-1, enables real-time detection of PCR amplified products. Amplification curves are shown in FIG. 5A. Human 18S rRNA from as little as 6 pg human genomic DNA from HaCat cells, equivalent to DNA from a single human cell, was detected. Changes of fluorescence intensity are shown in FIG. 5B.

FIGS. 6A-6B show PCR amplification of human genomic DNA with a nuclease free DNA polymerase.

Taq DNA polymerase consists of two separate domains, i.e, a 5′nuclease domain located at the N-terminus and a DNA polymerase domain located at the C-terminus. Deletion of its nuclease domain completely removes its 5′ endo/exonuclease activity. KlenTaq and Stoffel DNA polymerase were derived from Taq DNA polymerase with 5′ nuclease domain removed. Higher thermostability and higher replication fidelity than Taq DNA polymerase were reported (Lawyer, 1993; Barnes, 1992). Inclusion of a structure specific nuclease, Afu FEN-1, in PCR makes it possible to do simultaneous amplification and detection. A fragment of human 18S ribosomal RNA gene from HaCat cell genomic DNA was amplified with Stoffel DNA polymerase. Amplification curves are shown in FIG. 6A. A standard curve (FIG. 6B) was generated with a 10-fold serially diluted human genomic DNA ranging from 6 pg to 6,000 pg per reaction. Efficiency of PCR amplification, calculated from value of slope, is about 100%.

FIGS. 7A-7B show cleavage of Afu Fen-1 of various structures.

FIGS. 8A-8B show CE detection of cleaved products—Bst DNA polymerase LF and Afu FEN-1.

Cleavage of probe results in changes in physical, chemical and biological properties of the probe molecule, such as size, charge, mobility etc. One way to detect cleavage is to monitor mobility of fluorophore bearing fragments on capillary electrophoresis (CE). There is only one peak observed with uncleaved probe (FIG. 8A). Primer extension by Bst DNA polymerase LF triggered cleavage of probe by Afu FEN-1 as evidenced by presence of multiple peaks (FIG. 8B).

FIGS. 9A-9C shows CE detection of cleaved products—Stoffel DNA polymerase and Afu FEN-1. FIG. 9A shows uncleaved probe. No template control did not produce any additional peak (FIG. 9B) while template plus reaction generated multiple cleaved products (FIG. 9C).

FIGS. 10A-10B shows CE detection of cleaved products—MMLV reverse transcriptase and Afu FEN-1. Single peak was seen with uncleaved probe (FIG. 10A). Primer extension by MMLV reverse transcriptase and probe cleavage by Afu FEN-1 generated a major cleaved product (FIG. 10B).

FIGS. 11A-11B shows CE detection of cleaved products—PCR with Stoffel DNA polymerase and Afu FEN-1. PCR amplification of 2.4 ng human genomic DNA with Stoffel DNA polymerase and Afu FEN-1 yielded multiple products (FIG. 11B). FIG. 11A shows uncleaved probe control.

### DETAILED DESCRIPTION

The present invention is directed, in part, to using structure specific nuclease alone or in combination with nucleic acid polymerase for generating signals indicative of the presence of target nucleic acid sequences and for detection and measurement of target nucleic acid sequences.

According to the present invention, a signal indicative of the presence of a target nucleic acid sequence in a sample can be generated by incubating the sample with a structure specific nuclease and a suitable probe. Alternatively, a signal indicative of the presence of a target nucleic acid sequence in a sample can be generated by incubating the sample with a structure specific nuclease and a suitable probe and amplifying, e.g., concurrently a sequence containing the target nucleic acid sequence with a nucleic acid polymerase and, e.g., at least one amplification primer. The generated signals can be used to detect or measure the amount of target nucleic acid sequences in a sample.

A probe suitable for the present invention can be any oligonucleotide containing a signaling moiety and a sequence capable of specific hybridization to a desired target nucleic acid sequence. The probe of the present invention can also include one or more moieties to facilitate hybridization of the probe to a target nucleic acid sequence, increase cleavage of the probe by the structure specific nuclease of the present invention, increase detection of the probe, and reduce background noise. For example, the probe of the present invention can also contain certain conjugations, e.g., minor groove binder, pyrene, cholesterol, acridine, and biotin to facilitate amplification and/or detection.

The probe of the present invention can contain any natural bases including, without any limitation, adenine, N⁶-methyl adenine, N⁶-isopentenyl adenine, guanine, 7-methyl guanine, queuosine, wyosine, inosine, cytosine, 3-methyl cytosine, 5-methyl cytosine, uracil, dihydrouracil, pseudouracil, 4-thiouracil, and thymine. The probe of the present invention can also contain any base analogs including, without any limitation, 7-deaza-adenine, 7-deaza-guanine, 2-amino purine, 2,6-diamino purine (adenine and guanine), 2- and/or 6-thio purine (adenine and guanine), 5-bromo uracil, 5-nitro indole, 5-propynyl uracil, iso-cytosine, iso-guanine 5-phenyl-uracil, 2-N-methylguanine, 5-butynyl-uracil, dimethylthiazole uracil, 5-propynyl cytocine, 5-phenyl-cytosine, 5-butynyl-cytosine, dimethylthiazole cytosine, 9-(aminoethoxy) phenoxazine, 5-(N-aminohexyl) carbamoyl-uracil, 6-azathymine, N²-imidazolylpropyl-2-amino adenine, and N²-imidazolylpropyl-guanine.

The backbone of the probe of the present invention can include either a regular 5′ to 3′ phosphodiester bond or various modifications thereof. Examples of suitable modifications include peptide linkage as seen in peptide nucleic acid (PNA), phosphorothioate, N³′-->P5′ phosphoramidate, inverted linkage, methylphosphonate, morpholino nucleic acid, boranophosphonate, phosphoro-N-butylamidates, and methylenemethylimines. The sugar group of the probe used in the present invention usually is ribose and/or 2-deoxyribose. In one embodiment, the probe of the present invention contains one or more other types of sugar moieties, e.g., 2-O-alkyl ribose, arabinose, 2-deoxy arabinose, 2-deoxy-2-fluoro arabinose, 1,5-anhydro hexitol, 2-O,4-C-methylene ribose, and cyclohexene.

According to the present invention, the probe of the present invention contains a sequence which is capable of specific hybridization to a desired target nucleic acid sequence. In one embodiment, the probe of the present invention contains a sequence complementary to a target nucleic acid sequence. In another embodiment, nucleotides non-complementary to target sequences are located within or at either or both ends of the probe of the present invention, e.g., a 5′ flap or 3′ flap or both, provided that the probe/target nucleic acid duplex still has sufficient specificity and thermostability at cleavage temperature. In yet another embodiment, the probe of the present invention does not have a secondary structure that changes, e.g., diminishes or disappears upon hybridizing of the probe to a target nucleic acid sequence. In still another embodiment, the probe of the present invention does not have a 5′ flap, 3′ flap or both.

The length and selection of the probe's complementary region are determined by multiple factors well known to one skilled in the art. For example, in designing a suitable probe for the present invention one usually needs to consider the base composition, sequence context, size and sequence of amplified region, cleavage temperature, thermostability of probe/target duplex, hybridization stringency, assay specificity requirement, degeneracy, and the presence of modification group(s) etc. In general, the length of nucleotides in the probe complementary to a target nucleic acid sequence ranges from 5 to 500 nucleotides, 8 to 40 nucleotides, or 10 to 30 nucleotides, contiguously or in aggregate.

In one embodiment, the probe of the present invention is not extendable by nucleic acid polymerase. Since a 3′ hydroxy group of a basepairing nucleotide is essential for oligonucleotide extension, non-extendability can be achieved by elimination or modification of the 3′ hydroxy group. Any means suitable for modification of the 3′ hydroxy group to prevent polymerase extension can be used. For example, the 3′ hydroxy group of the probe can be modified to produce 2′,3′-dideoxynucleotide, 3′ phosphate, 3′ alkylation, acyclic nucleotide at the 3′ end of the probe. The 3′ end of the probe of the present invention can also include one or more nucleotides that are not complementary to the 5′ end of a target sequence to prevent efficient extension by a nucleic acid polymerase. Other modifications which interfere with nucleic acid polymerase binding to the 3′ end or inhibit the probe's 3′ end activity in polymerase extension can also be incorporated into the probe.

According to the present invention, the probe of the present invention includes a signaling moiety, which can be any moiety that is inactivated when the probe is not hybridized to a target nucleic acid sequence, but capable of forming a suitable substrate for a structure specific nuclease to generate a signal when the probe is hybridized to a target nucleic acid sequence. The generated signal can be any signal that is suitable for detection. For example, the generated signal can be a signal detectable by physical, chemical, photochemical, immunochemical, and biochemical methods including, but not limited to fluorescence, chemiluminescence, bioluminescence, electrochemiluminescence, phosphorescence, time-resolved spectrometry, fluorescence polarization, enzymatic reaction, radioactivity, colorimetry, mass spectrometry, magnetism, electrophoretic mobility, and chromatography.

In one embodiment, the signaling moiety includes an indicating moiety and a regulating moiety separated by a site susceptible to the cleavage of a structure specific nuclease. The indicating moiety is inhibited by the regulating moiety when the probe is not hybridized to a target nucleic acid sequence, while the indicating moiety is separated from the regulating moiety or released from the inhibition of the regulating moiety by the cleavage of a structure specific nuclease when the probe is hybridized to a target nucleic acid sequence.

In another embodiment, the signaling moiety includes a labeled moiety and a quencher moiety which quenches the labeled moiety when the probe is not hybridized to a target nucleic acid sequence. Such interaction between the labeled moiety and the quencher moiety can be between any suitable entities including without any limitation small molecules, e.g., fluorophores and their quenchers and large molecules, e.g., protein molecules (Boute, 2002). Such interaction can also be based on any suitable mechanism. For example, the labeled moiety and the quencher moiety can interact with each other based on resonance energy transfer including without any limitation fluorescence resonance energy transfer (FRET), luminanscence resonance energy transfer (LRET), phosphorescence resonance energy transfer (PRET), and bioluminescence resonance energy transfer (BRET).

Among various labeled moieties and quencher moieties, FRET probes have been widely used in detection of amplified target molecules. The most common FRET probe has two interactive moieties. One is fluorescence donor group and the other one is fluorescent acceptor group. Although the acceptor group can be fluorescent, it is preferred to have a non-fluorescent group as the acceptor. The donor group can be placed either at the 5′ end, in the middle, or at the 3′ end. So can the acceptor group. It is preferred to put the donor group at 5′ end. Cleavage of probe by a structure specific nuclease when the probe hybridizes to a target nucleic acid will separate the donor group from the acceptor group and release quenching of the donor's fluorescence by the acceptor.

The signaling moiety of the probe of the present invention can also have more than two interactive moieties. For example, U.S. Pat. No. 5,952,180 discloses a method to make an extendable oligonucleotide with a distinguishable fluorescence emission spectrum. The distinguishable fluorescence emission spectrum is generated by a combinatorial fluorescence energy transfer tag. Such technology can be used to make a non-extendable FRET probe for the present invention. Another example relates to a wavelength-shifting probe with three interactive groups as disclosed in U.S. Pat. No. 6,037,130.

In yet another embodiment, oligonucleotide itself can be a regulating moiety that interacts with an indicating moiety. For example, Nurmi has reported synthesis of a singly labeled fluorescent terbium chelate probe and its use in detection of PCR products (Nurmi, 2000). Fluorescence of terbium chelate attached to an oligonucleotide can be quenched by the oligonucleotide itself. Separation of terbium chelate from the oligonucleotide by a structure specific nuclease releases such quenching.

In general, the probe of the present invention is unimolecular. Nevertheless the probe of the present invention can also be binary or trinary. For example, the probe of the present invention can be a binary oligonucleotide prepared according to the methods disclosed in U.S. Pat. No. 6,432,642. Another binary probe containing two complementary oligonucleotides is described in Li et al. (Li, 2002). The probe of the present invention can also be a tripartite molecule prepared by the methods described for making a tripartite molecular beacon. (Nutiu, 2002).

In addition, the probe of the present invention can be used to generate signals for one or more target nucleic acid sequences. For example, two probes of the present invention can be used to detect two different target nucleic acid sequences, with each probe having an indicating moiety different from the other probe. Preferably the detection of one indicating moiety does not interfere with the detection of the other indicating moiety. The generation and detection of signals for each target nucleic acid sequence usually can be carried out simultaneously or subsequent of each other.

According to the present invention, a structure specific nuclease can generate a signal when the probe of the present invention hybridizes to a target nucleic acid sequence. The structure specific nuclease of the present invention can be any nuclease having a 5′ endo/exonuclease activity and cleaves nucleotide(s) in a structure specific manner, e.g., cleaves nucleotide(s) off the probe of the present invention when the probe hybridizes to a target DNA or RNA sequence. In one embodiment, the structure specific nuclease of the present invention is capable of cleaving the probe of the present invention when it is hybridized to an RNA sequence. For example, Taq DNA polymerase mutants and Tth DNA polymerase mutants have a significant RNA template dependent 5′ nuclease activity (Ma, 2000; Eis, 2001). Those enzymes can be used in processes wherein generation of RNA sequences is involved, e.g., in NASBA, TMA, or 3SR amplification process.

In another embodiment, the structure specific nuclease of the present invention has none or minimum nuclease activity towards single strand oligonucleotides, e.g., natural or denatured single strand oligonucleotides. In yet another embodiment, the structure specific nuclease of the present invention is a flap endonuclease-1 (FEN-1). Many FEN-1s have been isolated, purified, and characterized. Genes encoding FEN-1 have also been cloned for producing FEN-1. Examples of FEN-1s suitable for the present invention include without any limitation FEN-1s from Archaeglobus fulgidus, human, Methanobacterium thermoautotrophicum ΔH, Methanococcus jannaschii, murine, Pyrococcus furiosus, Pyrococcus horihoshii, Pyrococcus woesei, Xenopus laevis, calf RTH-1 protein, Rad27 protein of Sacchromyces cerevisiae, and Rad2 protein of Schizosacchromyces pombe. FEN-1 homologs that still retain whole or partial 5′ endonuclease activity can also be used for the present invention. For example, RNase H from T4 phage, D15 exonuclease from T5 phage, Gene6 exonuclease from T3 and T7 phage have been shown to have FEN-1 activity, thus are suitable for the present invention.

In still another embodiment, the structure specific nuclease of the present invention is a polymerase, e.g., a DNA polymerase which has 5′ endo/exonuclease activity, but minimum polymerizing activity or an entity containing a 5′ endo/exonuclease domain of a DNA polymerase, but with minimum polymerizing activity. It has been shown that the 5′ endo/exonuclease domain of various DNA polymerases can function independently of the other domains, e.g., can function without the presence of the polymerizing domain. For example, 5′ endo/exonuclease activity associated with eubacterial DNA polymerases from Bacillus caldotenax, Deinococcus radiodurans, Escherichia coli (DNA polymerase I), Streptococcos pneumoniae, Thermus aquaticus, Thermus brokianus, Thermus caldophilus GK24, Thermus flavus, Thermus rubens, and Thermus thermophilus has been shown to function properly without the presence of DNA polymerizing activity, thus can be used for the present invention.

In still yet another embodiment, the structure specific nuclease of the present invention is an RNAase, e.g. RNAase H, which is capable of recognizing RNA-DNA hybrids and cleaving RNA nucleotides in the hybrids or nucleotides with ribose as sugar groups. Such RNAase can be used with a probe of the present invention containing RNA nucleotides or nucleotides with ribose as sugar groups within or at either end of the probe.

An entity having a 5′ endo/exonuclease activity, but minimum polymerizing activity can be naturally existing or obtained by any suitable means available to one skilled in the art. For example, mutants generated by site-directed mutation(s) or deletions that eliminate DNA polymerizing function have been shown to still retain 5′ nuclease activity (Lyamichev, 1999; Kaiser, 1999), thus can be used as the structure specific nuclease for the present invention.

According to the present invention, the probe of the present invention is capable of hybridizing to a target nucleic acid sequence to form a suitable substrate for the structure specific nuclease of the present invention. A suitable substrate for the structure specific nuclease of the present invention includes any nucleic acid structure recognizable and cleavable by the structure specific nuclease. Examples of various suitable substrates for the structure specific nuclease are illustrated in FIG. 1. Usually suitable substrates for the structure specific nuclease include a nucleic acid double-strand duplex or base pairing structure. Such nucleic acid double-strand duplex or base pairing structure can be a DNA-DNA duplex or DNA-RNA duplex and can also include non-complementary bases within or at either end or strand of the duplex or base pairing structure, e.g., 5′ flap or 3′ flap.

In one embodiment, a suitable substrate for the structure specific nuclease includes a nucleic acid double-strand duplex of at least one, two, or three contiguous base pairs. In another embodiment, a suitable substrate for the structure specific nuclease includes a first probe of the present invention hybridized to a target nucleic acid sequence and a second probe hybridized to the 5′ upstream of the target nucleic acid sequence. For example, the first probe of the present invention can hybridize to a target nucleic acid sequence to form the first nucleic acid double-strand duplex with or without a 5′ flap, 3′ flap, or both while the second probe can hybridize, with or without a 5′ flap, 3′ flap, or both, to the 5′ upstream of the target nucleic acid sequence with a gap, a nick, or an overlap with respect to the first nucleic acid double-strand duplex.

A gap or a nick between the first nucleic acid double-strand duplex and the second probe means that the second probe hybridizes to a region 5′ upstream of the target nucleic acid sequence and the complementary or hybridized region is at least one nucleotide apart from or juxtaposed to the target nucleic acid sequence. An overlap between the first nucleic acid double-strand duplex and the second probe means that the second probe hybridizes to a region 5′ upstream of the target nucleic acid sequence and the complementary or hybridized region has at least one nucleotide overlap with the target nucleic acid sequence.

In yet another embodiment, a suitable substrate for the structure specific nuclease includes a first probe of the present invention hybridized to a target nucleic acid sequence, e.g., to form the first double-strand duplex and an amplification primer hybridized to the 5′ upstream of the target nucleic acid sequence and being extended by a nucleic acid polymerase of the present invention wherein the extension end is at least one nucleotide away from, juxtaposed, or at least one nucleotide overlap with the first double-strand duplex.

According to the present invention, generating or detecting a signal indicative of the presence of a target nucleic acid sequence can be carried out by a structure specific nuclease alone or in the present of amplifying a sequence containing the target nucleic acid sequence. Such amplification process usually includes a nucleic acid polymerase and at least one or two amplification primers. It can be any type of amplification process suitable for the present invention, e.g., linear amplification, PCR, or isothermal, etc.

The nucleic acid polymerase of the present invention can be any nucleic acid polymerase, e.g., DNA or RNA polymerase that does not have any substantial 5′-3′ nuclease activity, e.g., does not have any intrinsic 5′-3′ nuclease activity. For example, the nucleic acid polymerase of the present invention can be a reverse transcriptase, RNA polymerase, or DNA polymerase, with 3′-5′ exonuclease or proofreading activity and preferably with none or reduced 5′-3′ nuclease activity, e.g., reduced to an extent that no detectable signal could be generated in the absence of a structure specific nuclease. Reduction of 5′-3′ nuclease activity can be achieved usually by deletion, random mutation or site-directed mutagenesis. For example, reduction of 5′-3′ nuclease activity has achieved in Klenow fragment of E. coli DNA polymerase I, KlenTaq (Barnes, 1992), Stoffel DNA polymerase (Lawyer, 1993), AmpliTaq FS which is a point mutation of Taq DNA polymerase, R25C and R74H mutants (Merkens, 1995).

In one embodiment, the nucleic acid polymerase of the present invention includes nucleic acid polymerases isolated or cloned from a mesophilic, thermophilic, or hyperthermophilic hosts. In another embodiment, the nucleic acid polymerase of the present invention includes a DNA or RNA polymerase and an inhibitor or inhibiting domain which reduces the 5′-3′ nuclease activity of the polymerase.

In another embodiment, a DNA polymerase with strong strand displacement activity, e.g., Bst DNA polymerase large fragment is used in SDA, RCA, LAMP, SPIA™ or X-SPIA™ amplification process. In yet another embodiment, a DNA polymerase with reverse transcriptase activity is used in NASBA, 3SR, or TMA amplification process. Such polymerase includes reverse transcriptases identified in retroviruses such as Moloney murine leukemia virus (MMLV), Rous sarcoma virus (RSV), avian myeloblastosis virus (AMV), Rous Associated Virus (RAV), human immunodeficiency virus (HIV). In addition, many genetically engineered mutants of reverse transcriptase can be used for the present invention. Examples of mutant reverse transcriptase suitable for the present invention are SuperScript (Invitrogen), SuperScript II (Invitrogen), SuperScript III (Invitrogen), ThermoScript (Invitrogen), PowerScript (Clontech/BD Biosciences), StrataScript (Stratagene), Omniscript RT (Qiagen), Sensiscript (Qiagen), Expand RT (Roche Applied Science). Some DNA polymerases, such as those from Thermus thermophilus and carboxydothermus hydrogenformans, also have reverse transcriptase activity.

In still another embodiment, an RNA polymerase is used for NASBA, 3SR, or TMA amplification process. RNA polymerases suitable for such amplification process include without any limitation RNA polymerases from SP6, T4 and T7 bacteriophages.

In general, a thermostable DNA polymerase is used for PCR. Many thermostable DNA polymerases have been cloned and are suitable for the present invention. For example, thermostable DNA polymerases from Aeropyrum pernix, Aquifex aeolicus, Bacillus caldotenax, Methanobacterium thermoautotrophicum ΔH, Methanococcus jannaschii, Methanothermus fervidus, Pyrobaculum islandicum, Pyrococcus endeavori, Pyrococcus furiosus, Pyrococcus horihoshii, Pyrococcus profundus, Pyrococcus woesei, Pyrodictium occultum, Sulfolobus acidocaldarius, Sulfolobus solfataricus, Thermoanaerobacter thermohydrosulfuricus, Thermococcus celer, Thermococcus fumicolans, Thermococcus gorgonarius, Thermococcus kodakaraensis KOD 1, Thermococcus litoralis, Thermococcus peptonophilus, Thermococcus sp.9°N-7, Thermococcussp.TY, Thermococcus stetteri, Thermococcus zilligii, Thermoplasma acidophilum, Thermus aquaticus, Thermus brokianus, Thermus caldophilus GK24, Thermus flavus, Thermus rubens, Thermus thermophilus, and Thermotoga maritime can be used for the present invention.

According to the present invention, the structure specific nuclease of the present invention can be used either in the presence or absence of an amplification process, e.g., a nucleic acid polymerase. The structure specific nuclease of the present invention is usually compatible with an amplification process, e.g., a nucleic acid polymerase. In one embodiment, the structure specific nuclease and nucleic acid polymerase of the present invention are provided as one recombinant fusion protein. In another embodiment, the structure specific nuclease and nucleic acid polymerase are provided as separate recombinant protein.

Selection for structure specific nuclease most optimal for a particular amplification process or nucleic acid polymerase used for an amplification process is well within the ability of one skilled in the art. Usually the factors to consider in selecting compatible structure specific nuclease and nucleic acid polymerase include, without any limitation, reaction temperature requirement and thermostability of nuclease, compatibility of thermostability of the two enzymes, buffer compatibility of the two enzymes, template type, cleavage structure type, and fidelity requirement for target amplification.

According to another feature of the present invention, it provides a kit useful for generating and/or detecting a signal indicative of the presence of a target nucleic acid sequence. In one embodiment, the kit of the present invention comprises a probe and a structure specific nuclease of the present invention. In another embodiment, the kit of the present invention includes a probe, a structure specific nuclease, and a nucleic acid polymerase of the present invention. In yet another embodiment, the kit of the present invention includes a probe, a structure specific nuclease, a nucleic acid polymerase of the present invention and any other agent useful for an amplification process, e.g., amplification primer(s), buffer reagent(s), etc.

The present invention can be used in association with many amplification schemes, e.g., any amplification process in which single stranded nucleic acid molecules containing one or more target nucleic acid sequences are generated. It can be used in isothermal amplification such as NASBA, TMA, 3SR, RCA, SDA, LAMP, SPIA™ and X-SPIA™ as well as polymerase chain reaction which is a thermocycling amplification method.

To better understand how the present invention could be used in association with amplification methods, the present invention is illustrated with respect to each of the currently available amplification methods.

Use of the Present Invention in Association with NASBA, TMA, and 3SR

Due to their similarity in working principle, these methods are categorized and discussed in one group. They are used primarily to amplify RNA target at a constant temperature. Amplification comprises following steps:

i. RNA Template Directed Enzymatic Synthesis of Complementary DNA (cDNA).

This process is also called reverse transcription and the enzyme used is reverse transcriptase (RT). Reverse transcription is done with a first oligonucleotide with target sequence at 3′ region and a promoter sequence for an RNA polymerase at 5′ region. Single stranded promoter sequence is not transcriptionally functional until it becomes double stranded. An RNA/DNA heterduplex is formed as result of the reverse transcription.

Commonly used RTs are MMLV-RT, AMV-RT, RSV-RT, and their mutants. Some DNA polymerases, e.g, Tth DNA polymerase and C. therm. DNA polymerase, also possess RT activity. In addition to RNA template directed DNA polymerase activity, RT also can catalyze DNA template directed DNA synthesis.

ii. RNase H Degradation of RNA Strand in RNA/DNA Heteroduplex.

Many RTs also have RNase H activity which selectively degrades RNA strand in RNA/DNA heteroduplex. To proceed this step, the required RNase H activity is either provided by the reverse transcriptase or a separate RNase H. As a result of removal of RNA strand, cDNA becomes substantially single stranded.

iii. Synthesis of Double Stranded DNA.

A second oligonucleotide hybridizes to the single stranded cDNA and is extended by the reverse transcriptase to generate a DNA/DNA duplex. Now promoter region for the corresponding RNA polymerase is double stranded and functional.

iv. Synthesis of Single Stranded RNA by In Vitro Transcription.

With a functional promoter and an RNA polymerase, each DNA/DNA duplex generates hundreds of RNA molecules. It completes a cycle of amplification. As a result, each RNA template molecule is amplified hundreds of times.

v. Repeats of Steps i to iv.

This will amplify target nucleic acid exponentially.

To apply the present invention to NASBA, TMA, and 3SR, both single stranded cDNA generated in step ii and single stranded RNA in step iv can be utilized to form a cleavage structure with a labeled probe. Cleavage structure can be formed by the two only or with another oligonucleotide which is at least partially complementary to either of the single stranded nucleic acids and is upstream of the labeled probe. If the single stranded DNA is chosen to form cleavage structure, said another oligonucleotide can be the second oligonucleotide or the extended second oligonucleotide. If said single stranded RNA is chosen to form cleavage structure, said another oligonucleotide can be the first oligonucleotide or the extended first oligonucleotide. FIG. 1B (gapped structure), 1C (nicked structure), and 1D (overlapped structure) are examples of possible cleavage structures. Alternatively a third oligonucleotide can be included to form cleavage structures shown in FIG. 1E, 1H (gapped structures), 1F, 1I (nicked structures), 1G and 1J (overlapped structures).

It is preferred to form a cleavage structure with the single stranded DNA. When a cleavage structure is formed with the single stranded RNA, two kinds of cleavage could happen. One is wanted cleavage of probe molecule by the structure specific nuclease and the other is unwanted cleavage of RNA molecule by the RNase H activity. The latter destroys template molecules or amplified products and can slow down amplification dramatically. Co-existence of these two events will complicate the amplification. A cleavage structure with the single stranded DNA avoids this problem completely.

Application of Present Invention in RCA

RCA is an isothermal method to amplify either target nucleic acid and/or a reporter sequence. Both linear and exponential RCA schemes have been reported. Its applications include: i. detection of both DNA and RNA via signal or target amplification; ii. immunoassay with RCA using oligonucleotide conjugated antibody to detect protein molecules; iii. plasmid amplification for DNA sequencing; iv. in situ detection of a specific sequence.

For target nucleic acid detection, a 5′ phosphorylated single stranded DNA oligonucleotide contains target complementary regions at its 5′ end and 3′ end. Upon hybridization to the target template, 5′ end and 3′ end are separated by a nick. The nick is subsequently sealed by a DNA ligase to generate a circle DNA molecule. After binding to the single stranded DNA circle, a first oligonucleotide is extended by a DNA polymerase with strong strand displacement activity. Each hybridized primer can generate a linear single stranded DNA molecule with up to 10⁵ tandemly repeated concatemerized complementary strand of the DNA circle (Lizardi, 1998). This is linear form of RCA. With a second oligonucleotide which is homologous to the DNA circle and complementary to the extended first oligonucleotide, exponential amplification can be achieved.

Bst DNA polymerase LF, phage Φ129 DNA polymerase, exo⁻Vent DNA polymerase, Sequenase v2.0 have been reportedly used in RCA. Bst DNA polymerase LF performs best in exponential amplification by RCA (Lizardi, 1998).

In linear RCA, the single stranded concatemer can be used to form a bi-molecular cleavage structure with a probe. In a preferred embodiment, a tri-molecular cleavage structure is formed with addition of a non-extendable oligonucleotide (FIG. 1E-1J). Cleavage event can happen repeatedly. Consequently signal amplification is achieved. This provides a way to conduct template amplification and signal amplification in a single tube. With 10⁵ template amplification (Lizardi, 1998) and 3×10³ folds signal amplification (Lyamichev, 1999), the combined amplification power can be over 3×10⁸.

To apply the present invention to exponential RCA, both extended single stranded first and second oligonucleotide can be a candidate for forming a cleavage structure with a labeled probe and, if necessary, a third oligonucleotide. Exemplified cleavage structures are depicted in FIG. 1.

Application of Present Invention in SDA

SDA is an isothermal exponential amplification method which relies on DNA strand displacement. It utilizes two enzymes, namely a restriction endonuclease and a DNA polymerase, and two oligonucleotides. Each of the two oligonucleotides contains: (a) a tag sequence; (b) an unmodified sequence recognized by the restriction endonuclease; (c) complementary sequence to a double stranded target. SDA consists of following steps:

i. Extension of the two hybridized primers by the DNA polymerase incorporates a 2′-deoxyribosenucleoside-5′-o-(1-thiotriphosphate) and creates two hemiphosphorothioate restriction endonuclease recognition sites.

ii. The restriction endonuclease nicks the unmodified strands of the two hemiphosphorothioate recognition sites.

iii. Extension of the 3′ends at the nicks displaces the downstream strands and produces two single stranded DNA molecules.

iv. The two newly generated single stranded DNA undergo next cycle of amplification.

Although the original SDA was accomplished with a mesophilic DNA polymerase, i.e., exonuclease deficient Klenow fragment of E. coli DNA polymerase I (Walker, 1992), it has been replaced by a thermophilic SDA system which consists of a thermophilic DNA polymerase, Bst DNA polymerase LF (Spear, 1997). Advantages of the thermophilic system over the original system are higher specificity and faster amplification kinetics.

The present invention in SDA can be easily applied to SDA. Either of the two single stranded DNA can be used to form one or more cleavage structures depicted in FIG. 1.

When linear SDA is carried out, it produces single stranded DNA molecules which can be used to form a cleavage structure and generate detectable signal with the present invention. Signal amplification can also be achieved in the same way for RCA.

Application of Present Invention in SPIA and X-SPIA™

SPIA (Single Primer Isothermal Amplification) by Nugen Technologies is an isothermal amplification technology using method disclosed in U.S. Pat. No. 6,251,639. An RNA/DNA chimeric oligonucleotide binds to a target nucleic acid and gets extended by a DNA polymerase with strong strand displacement activity, such as Bst DNA polymerase LF. While RNA part of the extended RNA/DNA chimeric oligonucleotide is degraded by an RNase H, the DNA part remains hybridized to target nucleic acid. Extension of the remaining DNA sequence by the DNA polymerase displaces downstream DNA sequence and generate a single stranded DNA. Target sequence is amplified linearly when this process is repeated over and over again. Amplified product generated by SPIA is single stranded DNA. To apply the present invention to SPIA, a cleavage structure can be formed in the same way as that for linear RCA. It also provides a way to conduct template amplification, signal amplification and detection in one tube.

X-SPIA (exponential single primer isothermal amplification) is the exponential form of SPIA™. X-SPIA™ is accomplished by making complementary strand of the single stranded DNA produced by SPIA™. Like SDA, X-SPIA™ produces two single stranded DNA. The present invention can be applied to it as to SDA. Either or both of them can be used to form a cleavage structure(s).

Application of the Present Invention in LAMP

Four oligonucleotides and a DNA polymerase with strong strand displacement activity, e.g., Bst DNA polymerase LF, are employed in LAMP to amplify target nucleic acid sequence. In stage I Bst DNA polymerase LF extends two outer oligonucleotides and two inner oligonucleotides to form two partially single stranded dumb-bell like structures. Then in stage II the two inner oligonucleotides and the two dumb-bell like structures engage self-sustained exponential amplification. During the amplification, monomers and concatemers with various length of the two dumb-bell like structures are formed.

To apply the present invention to LAMP, those monomers and concatemers are used to form a cleavage structures with a probe (FIG. 1). Detectable signal is generated by cleavage of probe molecules within cleavage structures.

Application of the Present Invention in PCR

PCR is the most commonly used nucleic acid amplification technology. It involves thermocycling consisting of:
i. heat separation of double stranded DNA.
ii. annealing of two oligonucleotides to each single stranded DNA.
iii. extension of annealed oligonucleotides.
iv. repeating step i to iii.

So far there has not been a homogeneous detection system developed for PCR amplification with a high fidelity enzyme. The existing 5′ nuclease assay is incompatible with a high fidelity DNA polymerase. Even though Pfu DNA polymerase is of 8 fold higher fidelity than Taq DNA polymerase which is the most widely used enzyme in the 5′ nuclease assay (Lundberg, 1991), it has not been used in clinical nucleic acid testing. For applications like clinical testing, forensic use, food and hygene applications etc., faithfulness of amplification is of great consequences. Therefore it is needed to develop such a homogeneous detection system to accommodate use of high fidelity DNA polymerase in PCR.

The present invention can be applied to high fidelity PCR. Both heat denatured single stranded DNA can be used to form cleavage structures to further generate detectable signal.

### EXAMPLES

The following examples are intended to illustrate but not to limit the invention in any manner, shape, or form, either explicitly or implicitly. While they are typical of those that might be used, other procedures, methodologies, or techniques known to those skilled in the art may alternatively used.

### Example 1

### Design of Oligonucleotides

Oligonucleotide design was accomplished with software Oligo 4.02 published by National Biosciences Inc. An important factor affecting design of oligonucleotide is melting temperature (Tm) which is defined as the temperature at which 50% of oligonucleotide binds to its complementary oligonucleotide. The software gives three Tms based on different Tm prediction means. Nearest neighbor method is recognized for its best overall accuracy of Tm prediction. Therefore Tm by nearest neighbor method was adopted. Stable secondary structure and duplex structure especially 3′ duplexes should be avoided whenever it is possible.

To maximize signal generation, Tm of probe oligonucleotide should be significantly higher than Tm of primer oligonucleotides. An example of oligonucleotide design is listed in

TABLE 1

Table 1. Design of primers and probe for
PCR amplification of human 18S rRNA gene

| | |
|---|---|
| Sequence | Tm, ° C. |

| | | | | |
|---|---|---|---|---|
| Foward primer | 18SF | 5′-CGA GGC CCT GTA ATT GGA A-3′ | (SEQ ID NO. 1) | 65.1° C. |
| Reverse primer | 18SR | 5′-CGG CTG CTG GCA CCA GA-3′ | (SEQ ID NO. 2) | 68.7° C. |
| Probe | 18SPU | 5′-6FAM CGAGGA TCC ATT GGA | (SEQ ID NO. 3) | 74.1° C. |
| | | GGG*C*A*A*G BHQ1-3′** | | |

*phosphorothioate modification.
**Black Hole Quencher-1

### Example 2

### Homogeneous Sequence Detection by Bst DNA Polymerase LF and Afu FEN-1

Strand displacement is the most critical event in isothermal amplification processes SDA, RCA, LAMP, SPIA™ and X-SPIA™. A key component in these amplification methods is a DNA polymerase with strong strand displacement activity. Bst DNA polymerase LF is a genetically modified Bst DNA polymerase with its 5′ nuclease domain removed. It is the most widely used DNA polymerase in these methods. In all those isothermal amplification methods, single stranded DNA is generated. If reaction contains a template specific probe and a structure specific nuclease, the probe can bind to the single stranded DNA and be cleaved when an appropriate structure is formed. Therefore essential events are: i. generation of single stranded DNA; ii. hybridization of probe to the single stranded DNA; iii. formation of a cleavage structure by hybridization of a primer to the single stranded DNA and/or extension of the primer; and iv. cleavage of probe by a structure specific nuclease.

To demonstrate application of the invention disclosed herein in those isothermal amplifications, an experiment was performed as the following:
Nucleic acid polymerase: Bst DNA polymerase LF by New England Biolabs (8 Upper microliter)
Structure specific nuclease: Afu FEN-1

Oligonucleotides:

| | |
|---|---|
| Name | Sequence |

| | | |
|---|---|---|
| 18SF | 5′ CGA GGC CCT GTA ATT GGA A | (SEQ ID NO. 1) |
| | 3′ | |
| 18SPU | 5′ 6FAM CGAGGA TCC ATT GGA | (SEQ ID NO. 3) |
| | GGG*C*A*A*G BHQ1** | |
| 18SmT | 5′ CTT GCC CTC CAA TGG ATC | (SEQ ID NO. 4) |
| | CTC GTT AAA GGA TTT AAA GTG | |
| | GAC TCA TTC CAA TTA CAG GGC | |
| | CTC G 3′ | |

*phosphorothioate modification.
**Black Hole Quencher-1

All reactions contained 10 mM MOPS pH7.75, 10 mM KCl, 3 mM MgCl₂, 0.2 mM each dNTP (dATP, dCTP, dGTP, and TTP), 0.1% NP-40, 6% glycerol, 200 nM 18SF, 200 nM 18SP in a 20 μl reaction volume. One unit Bst DNA polymerase was used in all reactions except no enzyme control (NEC). No Afu FEN-1 was used in no enzyme control reaction and no nuclease control (NNC) reaction. Other reactions contained 5 to 20 ng Afu FEN-1. In no template control reactions, no 18SmT was included. 200 nM 18SmT was used in template⁺ reactions.

Reaction mixture was incubated 60° C. for an hour on ABI thermocycler 9600. Then 5 ul of 50 mM EDTA-Na₂, pH 8.0 was added to stop reaction. Analysis of results was done at 95° C. on ABI Prizm 7900HT. Because no probe could bind to the template at 95° C., any significant increase of 6FAM signal is an indication of probe cleavage by Afu FEN-1.

In the absence of template, no cleavage of free 18SP was seen even with 20 ng Afu FEN-1 (Table 2. and FIG. 2A). In the presence of template, a significant increase in 6FAM fluorescence in comparison to NEC and NNC was observed (FIG. 2B). Net changes in fluorescence change, ΔF=F_{template plus}−F_{template minus}, are shown in FIG. 2C. It demonstrated that the invention provides a way to detect a specific sequence with low background. A low background is critical for achieving high sensitivity of detection.

TABLE 2
Afu FEN-1 does not cleave single stranded probe.

| | NEC | NNC | 1 | 2 | 3 |
|---|---|---|---|---|---|
| Bst DNA pol. LF | 0 | IU | IU | IU | IU |
| Afu FEN-1 | 0 ng | 0 ng | 5 ng | 10 ng | 20 ng |
| Replicates | 3 | 3 | 3 | 3 | 3 |
| 6FAM Intensity | 2132 | 2048 | 2107 | 2107 | 2035 |
| Standard Deviation | 49 | 76 | 26 | 26 | 58 |

When linear amplification is carried out, a preferred cleavage structure is formed with an amplified product, a probe molecule, and a non-extendable oligonucleotide. Probe in this kind of cleavage structure has been demonstrated to be effectively degraded by structure specific nucleases.

In an exponential amplification scheme, a cleavage structure is preferably formed with an amplified product, a probe, an extendable oligonucleotide or an extended oligonucleotide. Two events could happen to the probe molecule within the cleavage structure. It could be either displaced by the extended upstream oligonucleotide or cleaved by Afu FEN-1. More Afu FEN-1 indeed improved cleavage (FIG. 2C). It is an indirect evidence that competition between the two events indeed exists. Although relative frequency of those two events was not determined, the data clearly demonstrated that sufficient cleavage occurred. Therefore the present invention is applicable to those amplification methods and provides a way to do homogeneous detection with no observable background.

### Example 3

### Real-time Detection of Strand Elongation by Bst DNA Polymerase LF

Nucleic acid Polymerase: Bst DNA Polymerase LF by New England Biolabs (8 Upper Micro Liter)
Structure specific nuclease: Afu FEN-1

Oligonucleotides:

| | |
|---|---|
| Name | Sequence |

| | | |
|---|---|---|
| 18SF | 5′ CGA GGC CCT GTA ATT GGA A | (SEQ ID NO. 1) |
| | 3′ | |
| 18SPU | 5′ 6FAM CGAGGA TCC ATT GGA | (SEQ ID NO. 3) |
| | GGG*C*A*A*G BHQ1** | |
| 18SmT | 5′ CTT GCC CTC CAA TGG ATC | (SEQ ID NO. 4) |
| | CTC GTT AAA GGA TTT AAA GTG | |
| | GAC TCA TTC CAA TTA CAG GGC | |
| | CTC G 3′ | |

*phosphorothioate modification.
**Black Hole Quencher-1

All reactions contained 10 mM MOPS pH7.75, 3 mM MgCl₂, 0.2 mM each dNTP (dATP, dCTP, dGTP, and TTP), 0.1% Tween-20, 6% glycerol, 60 nM Rox, 20 ng AfuFen-1, 200 nM 18SF, 200 nM 18SP in a 25 μl reaction volume. Various amount of Bst DNA polymerase LF, ranging from 0.25 units to 8 units per reaction, was included in the reactions.

Reaction mixture was incubated 60° C. for 45 minutes on ABI 7000. Fluorescence change was monitored at real-time. Results are shown in FIG. 3. Each data point is the average of four replicates.

Under the reaction condition, 1 unit Bst yielded the strongest signal. Below 1 unit enzyme per reaction, fluorescence increase was correlated with the amount of the enzyme used (FIG. 3A). Even in the absence of Bst DNA polymerase LF, AfuFen-1 generated significant signal that is about 20% of that by 1 unit Bst LF. This indicates that AfuFen-1 is capable to cleave hybridized probe.

Although the nuclease used herein is called flap endonuclease, a flap is not essential for such its structure specific cleavage as evidenced by lack of such one with the probe used in the experiment. Above 1 unit, kinetics of fluorescence increase was more complicated. Although fluorescence increase in reactions with 2 units enzyme initially was higher than that by 1 unit (FIG. 3B), the increase was surpassed later. Because Bst LF has strong strand displacement activity, there are at least two possible consequences by Bst LF mediated primer extension. One is to form a favorable cleavage structure and result in fluorescence increase. The other is to displace downstream probe. The consequence of such displacement is primer extension without increasing fluorescence intensity.

### Example 4

### Real-Time Detection of DNA Synthesis by Reverse Transcriptase

For the invention disclosed herein, generation of a single stranded nucleic acid by a nucleic polymerase is essential. In nucleic acid sequence amplification by NASBA, TMA, 3SR, there are two steps generating single stranded nucleic acid. First one is reverse transcription of an RNA molecule. Upon RNase H action, RNA strand in RNA/DNA duplex is degraded. Consequently the newly produced complementary DNA consists of significant portion of single stranded DNA. The current invention can be applied during copy of the single stranded DNA. Reverse transcriptase is responsible for synthesis of both DNA strands.

The second step is in vitro transcription which generates single stranded RNA molecules. With an appropriate probe and structure specific nuclease, single stranded RNA molecules can be used for sequence detection with the current invention as well. Application of the invention disclosed herein in NASBA, TMA, 3SR was exemplified in following experiment.

Nucleic acid polymerase: Moloney Murine reverse transcriptase (MMLV-RT) by Invitrogen (200 Upper microliter) Structure specific nuclease: Afu FEN-1 Oligonucleotides:

| | |
|---|---|
| Namen | Sequence |

| | | |
|---|---|---|
| 18SF | 5′ CGA GGC CCT GTA ATT GGA A | (SEQ ID NO. 1) |
| | 3′ | |
| 18SPU | 5′ 6FAM CGAGGA TCC ATT GGA | (SEQ ID NO. 3) |
| | GGG*C*A*A*G BHQ1** | |
| 18SmT | 5′ CTT GCC CTC CAA TGG ATC | (SEQ ID NO. 4) |
| | CTC GTT AAA GGA TTT AAA GTG | |
| | GAC TCA TTC CAA TTA CAG GGC | |
| | CTC G 3′ | |

*phosphorothioate modification.
**Black Hole Quencher-1

All reactions contained 20 mM MOPS pH7.50, 25 mM KCl, 4 mM MgCl₂, 0.5 mM each dNTP (dATP, dCTP, dGTP, and TTP), 0.1% Tween 20, 6% glycerol, 60 nM Rox, 200 nM 18SF, 200 nM 18SP, 100 unit MMLV-RT in a 25 μl reaction volume. Forty nanograms Afu Fen-1 and/or 200 nM 18SmT were included in some reactions. Reaction mixtures were incubated at 45° C. for 90 minutes on ABI 7000. Fluorescence change was monitored at real-time.

As shown in FIG. 4, both AfuFen-1 and template are required to generate detectable signal.

### Example 5

### Real-Time PCR Amplification of Human Genomic DNA with a High Fidelity Thermostable Enzyme

High fidelity nucleic acid amplification is critical for any amplification method. Any mis-incorporation of wrong nucleotide(s) can be exponentially amplified in later cycles and lead to qualitatively and/or quantitatively wrong results. This is especially critical for probe based detection method. DNA polymerases with 3′ to 5′ exonuclease activity which is also called proof-reading activity, such as Pfu DNA polymerase, Tli DNA polymerase, have been shown higher fidelity than those without such kind of nuclease activity, e.g., Taq DNA polymerase. There has not been any report on probe based real time PCR with a DNA polymerase having proof-reading activity. In an attempt to apply the current invention to high fidelity PCR amplification, the following experiment was done:
Nucleic acid polymerase: PfuTurbo DNA polymerase by Stratagene (2.5 Upper microliter). According to Stratagene, PfuTurbo DNA polymerase is a specially formulated Pfu DNA polymerase which has been know for its 8-fold higher fidelity than Taq DNA polymerase.
Structure specific nuclease: Afu FEN-1
Human genomic DNA: HaCat cell genomic DNA.

Oligonucleotides:

| | |
|---|---|
| Name | Sequence |

| | | |
|---|---|---|
| 18SF | 5′ CGA GGC CCT GTA ATT GGA A | (SEQ ID NO. 1) |
| | 3′ | |
| 18SR | 5′ CGG CTG CTG GCA CCA GA 3′ | (SEQ ID NO. 2) |
| 18SPU | 5′ 6FAM CGAGGA TCC ATT GGA | (SEQ ID NO. 3) |
| | GGG*C*A*A*G BHQ1** | |

*phosphorothioate modification
**Black Hole Quencher-1

The phosphorothioate modification is to inhibit non-specific probe degradation by 3′ to 5′ exonuclease activity of PfuTurbo DNA polymerase. Without this modification, such a non-specific degradation could remove quencher BHQ-1 from the probe. As the result of separation of BHQ-1 and 6FAM, there would be an increase in 6FAM signal which could interfere detection.

Twenty five microliter reaction mix contained 10 mM MOPS pH7.75, 3 mM MgCl₂, 0.2 mM each dNTP (dATP, dCTP, dGTP, and TTP), 0.1% NP-40, 6% glycerol, 200 nM 18SF, 200 nM 18SR, 200 nM 18SP, 5 ng Afu FEN-1 and 0.625 U PfuTurbo DNA polymerase. Various amount of HaCat genomic DNA ranging from 0 (no template control), 6 pg, 60 pg, 600 pg was included in reactions.

Real-time PCR was performed on ABI Prizm 7900HT with the following thermocycling parameters: 95° C., 1 min.-->(95° C., 15 sec.-->60° C., 1 min.) for 40 cycles. Data were collected at real-time and analyzed with software SDS version 2.0. (Applied Biosystems).

Amplification curves are shown in FIG. 5A. Ct values, which are useful for gene quantification, are shown in FIG. 5B. While different amount of genomic DNA template gave well separated Ct value, no template control (NTC) reactions did not generate any detectable signal (Ct=40). No detectable signal in NTC reaction is of significance in two aspects: i. demonstration of no cleavage of unhybridized single stranded probe by Afu FEN-1, and ii. phosphorothioate modification at the probe's 3′ end protects the probe from non-specific degradation by 3′ to 5′ exonuclease activity associated with PfuTurbo DNA polymerase. Amplification and detection are highly sensitive as evidenced by the fact that as little as 6 pg human genomic DNA, which is equivalent to the amount of DNA from a single cell, was successfully amplified and detected by the invention disclosed herein.

### Example 6

### Real-Time PCR Amplification of Human Genomic DNA with a Nuclease Free Thermostable Enzyme

Taq DNA polymerase consists of two domains: i. a 5′ exo/endonuclease domain located at the N-terminal region; and ii. a DNA polymerase domain in the C-terminal region. Deletion of the exo/endonuclease domain eliminates the nuclease activity completely. The truncated DNA polymerase has higher replication fidelity and higher thermostability than the wild type Taq DNA polymerase (Lawyer, 1993; Barnes, 1992). Improvements in these two aspects are very beneficial to PCR amplification. Because a non-extendable probe hybridized to PCR amplicon is inhibitory, it is practically very difficult to use molecular beacon with this kind of enzyme (Yu, 1997). The current invention can improve both amplification and real-time detection. An experiment was designed as following:
Nucleic acid polymerase: Stoffel DNA polymerase (10 U per microliter, Applied Biosystems) which is a 289 amino acid N-terminal deletion mutant of Taq DNA polymerase.
Structure specific nuclease: Afu FEN-1
Human genomic DNA: HaCat cell genomic DNA.

Oligonucleotides:

| | |
|---|---|
| Name | Sequence |

| | | |
|---|---|---|
| 18SF | 5′ CGA GGC CCT GTA ATT GGA A | (SEQ ID NO. 1) |
| | 3′ | |
| 18SR | 5′ CGG CTG CTG GCA CCA GA 3′ | (SEQ ID NO. 2) |
| 18SPU | 5′ 6FAM CGAGGA TCC ATT GGA | (SEQ ID NO. 3) |
| | GGG*C*A*A*G BHQ1** | |

*phosphorothioate modification.
**Black Hole Quencher-1

Twenty five microliter reaction mix contained 10 mM MOPS pH7.75, 3 mM MgCl₂, 0.2 mM each dNTP (dATP, dCTP, dGTP, and TTP), 0.1% NP-40, 6% glycerol, 200 nM 18SF, 200 nM 18SR, 200 nM 18SP, 5 ng Afu FEN-1 and 1.0 U Stoffel DNA polymerase. Various amount of HaCat genomic DNA ranging from 0 (no template control), 6 pg, 60 pg, 600 pg, 6,000 pg was included in reactions.

Real-time PCR was performed on ABI Prizm 7900HT with the following thermocycling parameters: 95° C., 1 min.-->(95° C., 15 sec.-->60° C., 1 min.) for 40 cycles. Data were collected at real-time and analyzed with software SDS version 2.0. (Applied Biosystems).

Amplification curves are shown in FIG. 6A. A standard curve is shown in FIG. 6B. Correlation coefficient of the standard curve is 1.00. Slope of the stand curve, a measure of amplification efficiency, is −3.29 which indicates that amplification efficiency was very close to 100%. As low as 6 pg human genomic DNA, which is equivalent to amount of DNA from a single human cell, was reliably detected.

### Example 7

### Cleavage structures of Afu Fen-1

AfuFen-1 is a member of flap endonuclease family. Previous studies have shown that many structures can be cleaved by flap endonucleases. For the invention disclosed herein, it is important to have a cleavage structure that can be efficiently cleaved by the flap endonuclease in the presence of a DNA polymerase. There are many ways a DNA polymerase can interact with a flap endonuclease. Three examples of the ways of interaction are: i. a DNA polymerase may interact with the flap endonuclease directly; ii. an elongated primer and a hybridized probe form a structure that can serve as a substrate for the flap endonuclease; iii. the DNA polymerase forms a complex with an elongated primer then interacts with the flap endonuclease.

Several cleavage structures were formed with oligos listed in the following table:

| | |
|---|---|
| Namen | Sequence |

| | | |
|---|---|---|
| 18SF | 5′ CGA GGC CCT GTA ATT GGA | (SEQ ID NO. 1) |
| | A 3′ | |
| 18S + 1 | 5′ GGA ATG AGT CCA CTT TAA | (SEQ ID NO. 5) |
| | ATC CTT TAA C 3′ | |
| 18S + 0 | 5′ TGG AAT GAG TCC ACT TTA | (SEQ ID NO. 6) |
| | AAT CCT TTA A 3′ | |
| 18S-1 | 5′ TTG GAA TGA GTC CAC TTT | (SEQ ID NO. 7) |
| | AAA TCC TTT A 3′ | |
| 18S-2 | 5′ ATT GGA ATG AGT CCA CTT | (SEQ ID NO. 8) |
| | TAA ATC CTT T 3′ | |
| 18SPU | 5′ 6FAM CGAGGA TCC ATT GGA | (SEQ ID NO. 3) |
| | GGG*C*A*A*G BHQ1** | |
| 18SmT | 5′ CTT GCC CTC CAA TGG ATC | (SEQ ID NO. 4) |
| | CTC GTT AAA GGA TTT AAA | |
| | GTG GAC TCA TTC CAA TTA | |
| | CAG GGC CTC G 3′ | |

*phosphorothioate modification.
**Black Hole Quencher-1

The 18SmT is a template to which the rest oligonucleotides can bind. 18SF, 18S+1, 18S+0, 18S−1, 18S−2 all bind to 5′ end of 18SPU and form following cleavage structures:
i. a duplex structure formed with 18SPU and 18SmT
ii. a large gap formed with 18SF, 18SPU and 18SmT
iii. a 2 nucleotide gap formed with 18S−2, 18SPU, and 18SmT
iv. a 1 nucleotide gap formed with 18S−1, 18SPU, and 18SmT
v. a nick formed with 18S+0, 18SPU, and 18SmT
vi. an overlap structure formed with 18S+1, 18SPU, and 18SmT

All reactions contained 10 mM MOPS, pH7.75, 3 mM MgCl₂, 0.1% Tween-20, 6% glycerol, 60 nM Rox, 5 ng AfuFen-1, 1 unit Stoffel DNA polymerase, 200 nM each oligo. In one set of reaction, 0.2 mM each dNTP was included in the reaction mixture. Reactions are carried out at 60° C. for 60 minutes on ABI 7000.

In the absence of dNTP, DNA polymerization could not occur. Under this circumstance, the overlapped structure gave highest fluorescence signal and the fastest cleavage kinetics (FIG. 7A). Structures with a nick or a small gap can be cleaved effectively by AfuFen-1. It is noteworthy that the duplex structure formed only with 18SPU and 18SmT was recognized by AfuFen-1 and signal intensity was about ⅓ of the maximum.

Inclusion of dNTP in the reaction system affected AfuFen-1 cleavage dramatically as evidenced by the altered cleavage kinetics (FIG. 7B). Although cleavage of the overlapped structure still generated the strongest signal, it was not as robust as that in the absence of dNTP. However inclusion of dNTP did not impact cleavage of 18SPU/18SmT duplex structure. Cleavage of structures with a nick, a 1-nucleotide gap, or a 2-nucleotide gap was indistinguishable from each other and was very similar to that of the gap structure formed with 18SF, 18SPU and 18SmT. This result is very significant because it showed that position of amplification primer is flexible and does not alter signal generation greatly.

It is not known the exactly structure cleaved by AfuFen-1 in the presence of DNA polymerization. Kinetics indicates that it is not the overlapped structure. In the presence of DNA polymerization, cleavage is not as robust as that of the overlapped structure in the absence of DNA polymerization. This effect can be well compensated by template amplification that increases number of templates exponentially.

### Example 8

### Capillary Electrophoresis Detection of Cleaved Products Sequence Detection by Bst DNA Polymerase LF and Afu FEN-1

Experiments in Example 2 to 7 were done in closed tubes. There are several important advantages with that format. However its fluorescence detection sensitivity is low because of high fluorescence background caused incomplete quenching of uncleaved probe. Its capability to simultaneously amplify and detect multiple targets is also limited by difficulty to resolve a complex emission spectrum. Separation of cleaved products prior detection will provide a way to reduce fluorescence background and get a simplified spectrum for each fraction.

Capillary electrophoresis (CE) has been commonly used in DNA sequencing and DNA fragment size analysis. It is highly sensitive and capable to handle multiple targets. As an exemplified way to do product separation and detection, CE was used to detect cleavage. An experiment was performed as the following:
Nucleic acid polymerase: Bst DNA polymerase LF by New England Biolabs (8 Upper micro liter)
Structure specific nuclease: Afu FEN-1

Oligonucleotides:

| | |
|---|---|
| Name | Sequence |

| | | |
|---|---|---|
| 18SF | 5′ CGA GGC CCT GTA ATT GGA A | (SEQ ID NO. 1) |
| | 3′ | |
| 18SPU | 5′ 6FAM CGAGGA TCC ATT GGA | (SEQ ID NO. 3) |
| | GGG*C*A*A*G BHQ1** | |
| 18SmT | 5′ CTT GCC CTC CAA TGG ATC | (SEQ ID NO. 4) |
| | CTC GTT AAA GGA TTT AAA GTG | |
| | GAC TCA TTC CAA TTA CAG GGC | |
| | CTC G 3′ | |

*phosphorothioate modification.
**Black Hole Quencher-1

In a 20 μl reaction volume it consisted of 15 mM MOPS pH7.75, 3 mM MgCl₂, 0.2 mM each dNTP (dATP, dCTP, dGTP, and TTP), 0.1% NP-40, 6% glycerol, 200 nM 18SF, 200 nM 18SP, 200 nM 18SmT, 1 unit Bst DNA polymerase and 40 ng Afu FEN-1. No enzyme control did not get any Bst DNA polymerase LF and Afu FEN-1.

Reaction mixture was incubated 60° C. for an hour on ABI thermocycler 9600. Five microliters of 50 mM EDTA-Na₂ (pH 8.0) was added to stop reaction. Products were diluted 500 folds (no enzyme control) and 100 folds (enzyme plus) respectively before running on ABI Prizm 310. Results were analyzed with software GeneScan® 2.1.

No enzyme control reaction only displayed a single peak (FIG. 8A) while enzyme plus reaction produced 6 more bands. Detection by capilary electrophoresis is indeed more sensitive than homogeneous detection on ABI Prizm 7900. Improvement in sensitivity is estimated at least 100 folds.

### Example 9

### Capilary Electrophoresis Detection of Cleaved Products: Sequence Detection by Stoffel DNA Polymerase and Afu FEN-1

In a 20 μl reaction volume it consisted of 10 mM MOPS pH7.75, 3 mM MgCl₂, 0.2 mM each dNTP (dATP, dCTP, dGTP, and TTP), 0.1% NP-40, 6% glycerol, 200 nM 18SF, 200 nM 18SP, 200 nM 18SmT, 1 unit Stoffel DNA polymerase (Applied Biosystems) and 10 ng Afu FEN-

1. No Template Control Did Not Contain 18SmT.

Oligonucleotides:

| | |
|---|---|
| Name | Sequence |

| | | |
|---|---|---|
| 18SF | 5′ CGA GGC CCT GTA ATT GGA A | (SEQ ID NO. 1) |
| | 3′ | |
| 18SPU | 5′ 6FAM CGAGGA TCC ATT GGA | (SEQ ID NO. 3) |
| | GGG*C*A*A*G BHQ1** | |
| 18SmT | 5′ CTT GCC CTC CAA TGG ATC | (SEQ ID NO. 4) |
| | CTC GTT AAA GGA TTT AAA GTG | |
| | GAC TCA TTC CAA TTA CAG GGC | |
| | CTC G 3′ | |

*phosphorothioate modification.
**Black Hole Quencher-1

Reaction mixture was incubated 60° C. for an hour on ABI thermocycler 9600. Five microliters of 50 mM EDTA-Na₂ (pH 8.0) was added to stop reaction. Products were diluted 500 folds before running on ABI Prizm 310. Results were analyzed with software GeneScan® 2.1.

Like no enzyme control (FIG. 9A), no template control reaction only displayed a single peak (FIG. 9B). This once again demonstrated that Afu FEN-1 does not cleave single stranded probe. In the presence of template, five cleaved products were observed (FIG. 9C).

### Example 10

### Capilary Electrophoresis Detection Of Cleaved Products: Sequence Detection by Moloney Murine Reverse Transcriptase and Afu FEN-1

In a 20 μl reaction volume it consisted of 10 mM MOPS pH7.50, 20 mM KCl, 4 mM MgCl₂, 0.5 mM each dNTP (dATP, dCTP, dGTP, and TTP), 0.1% NP-40, 6% glycerol, 200 nM 18SF, 200 nM 118SP, 200 nM 18SmT, 200 units Moloney murine reverse transcriptase (Invitrogen) and 20 ng Afu FEN-1.

Oligonucleotides:

| | |
|---|---|
| Name | Sequence |

| | | |
|---|---|---|
| 18SF | 5′ CGA GGC CCT GTA ATT GGA A | (SEQ ID NO. 1) |
| | 3′ | |
| 18SPU | 5′ 6FAM CGAGGA TCC ATT GGA | (SEQ ID NO. 3) |
| | GGG*C*A*A*G BHQ1** | |
| 18SmT | 5′ CTT GCC CTC CAA TGG ATC | (SEQ ID NO. 4) |
| | CTC GTT AAA GGA TTT AAA GTG | |
| | GAC TCA TTC CAA TTA CAG GGC | |
| | CTC G 3′ | |

*phosphorothioate modification.
**Black Hole Quencher-1

Reaction mixture was incubated at 45° C. for an hour on ABI thermocycler 9600. Five microliters of 50 mM EDTA-Na₂, pH 8.0 was added to stop reaction. Products were diluted 50 folds before running on ABI Prizm 310. Results were analyzed with software GeneScan® 2.1.

Unlike reactions in Examples 8 and 9, Moloney murine reverse transcriptase and Afu FEN-1 produced a major cleaved product (FIG. 10B). FIG. 8A shows uncleaved probe.

### Example 11

### Capilary Electrophoresis Detection of Cleaved Products: PCR Amplification of Human Genomic DNA

In a 20 μl reaction volume it consisted of 10 mM MOPS pH7.75, 3 mM MgCl₂, 0.2 mM each dNTP (dATP, dCTP, dGTP, and TTP), 0.1% NP-40, 6% glycerol, 200 nM 18SF, 200 nM 18SR, 200 nM 18SP, 2.4 ng HaCat cell genomic DNA, 1 unit Stoffel DNA polymerase (Applied Biosystems) and 10 ng Afu FEN-1.

Oligonucleotides:

| Name | Sequence | |
|---|---|---|
| 18SF | 5′ CGA GGC CCT GTA ATT GGA A 3′ | (SEQ ID NO. 1) |
| 18SR | 5′ CGG CTG CTG GCA CCA GA 3′ | (SEQ ID NO. 2) |
| 18SPU | 5′ 6FAM CGAGGA TCC ATT GGA GGG*C*A*A*G BHQ1 | (SEQ ID NO. 3) |

*phosphorothioate modification.
**Black Hole Quencher-1

PCR was carried out on ABI thermocycler 9600, (95° C., 20 sec, -->60° C., 1 min.) for 40 cycles. Five microliters of 50 mM EDTA-Na₂, pH 8.0 was added to stop reaction. Products were diluted 500 folds before running on ABI Prizm 310. Results were analyzed with software GeneScan® 2.1. As many as 8 cleaved products were observed.

It is noticeably interesting that each enzyme produced a unique cleavage pattern. Difference in cleavage pattern can be attributed to characteristics of each enzyme and probe behavior under specific reaction condition. Selective blockage of cleavage at certain sites should reduce number of cleaved products and facilitate simultaneous detection of multiple sequences. Modifications of backbone, carbohydrate, and base of oligonucleotide to protect it from being attacked by nuclease are known arts.

It is to be understood that description of the present invention with illustrations and examples is not intended to limit the present invention to those. Instead it is for showing principle of the present invention and exemplifying its applications. Many variations and modifications will become evident to those skilled in the art with this detailed disclosure. The present invention is to encompass all variations and modifications within the spirit and scope of the following claims.

### REFERENCES

U.S. PAT. DOCUMENTS

| | | | |
|---|---|---|---|
| 4,683,195 | July 1987 | Mullis et al | 435/6 |
| 4,683,202 | July 1987 | Mullis | 435/91 |
| 5,011,769 | April 1991 | Duck et al. | 435/6 |
| 5,130,238 | July 1992 | Malek et al | 435/91.21 |
| 5,210,015 | May 1993 | Gelfand et al. | 435/6 |
| 5,270,184 | December 1993 | Walker et al | 435/91.2 |
| 5,399,491 | March 1995 | Kacian et al | 435/91.21 |
| 5,403,711 | April 1995 | Walder et al. | 435/6 |
| 5,409,818 | April 1995 | Davey et al | 435/91.21 |
| 5,437,990 | August 1995 | Burg et al | 435/91.2 |
| 5,480,784 | January 1996 | Kacian et al | 435/91.21 |
| 5,487,972 | January 1996 | Gelfand et al. | 435/6 |
| 5,554,517 | September 1996 | Davey et al | 435/91.21 |
| 5,804,375 | September 1998 | Gelfand et al. | 435/6 |
| 5,837,450 | November 1998 | Dahlberg et al. | 435/6 |
| 5,846,717 | December 1998 | Brow et al. | 435/6 |
| 5,854,033 | December 1998 | Lizardi | 435/91.2 |
| 5,888,779 | March 1999 | Kacian et al | 435/91.2 |
| 5,916,779 | June 1999 | Pearson et al | 435/91.2 |
| 5,925,517 | July 1999 | Tyagi et al. | 435/6 |
| 5,952,180 | September 1999 | Ju | 435/6 |
| 5,985,557 | November 1999 | Prudent et al. | 435/6 |
| 5,994,056 | November 1999 | Higuchi | 435/6 |
| 6,001,567 | December 1999 | Brow et al. | 435/6 |
| 6,037,130 | March 2000 | Tyagi et al. | 435/6 |
| 6,063,603 | May 2000 | Davey et al | 435/91.2 |
| 6,090,543 | July 2000 | Prudent et al. | 435/6 |
| 6,090,591 | July 2000 | Burg et al | 435/91.2 |
| 6,171,785 | January 2001 | Higuchi | 435/6 |
| 6,183,960 | February 2001 | Lizardi | 435/6 |
| 6,210,884 | April 2001 | Lizardi | 435/6 |
| 6,214,979 | April 2001 | Gelfand et al. | 435/6 |
| 6,251,639 | Jun. 26, 2001 | Kurn | 435/91.2 |
| 6,344,329 | February 2002 | Lizardi | 435/6 |
| 6,348,314 | February 2002 | Prudent et al. | 435/6 |
| 6,350,580 | February 2002 | Sorge | 435/6 |
| 6,410,278 | June 2002 | Notomi et al. | 435/91.2 |
| 6,432,642 | August 2002 | Livak et al. | 435/6 |

### OTHER PUBLICATIONS

Applied Biosystems, 1994, Evaluating and isolating synthetic oligonucleotides
Bae et al., 1999, “Expression, purification, characterization and crystallization of flap endonuclease-1 from Methanococcus jannaschii” Mol. Cells 9(1): 45-48.
Barnes, 1992, “The fidelity of Taq polymerase catalyzing PCR is improved by an N-terminal deletion” Gene 112(1): 29-35.
Boute et al., 2002, “The use of resonance energy transfer in high-throughput screening: BRET versus FRET” Trends in Pharmacol. Sci. 23(8): 351-354.
Cariello et al., 1991, “Fidelity of Thermococcus litoralis DNA polymerase (Vent™) in PCR determined by denaturing gradient gel electrophoresis” Nucleic Acids Res. 19(15): 4193-4198.
Eis et al., 2001, “An invasive cleavage assay for direct quantitation of specific RNAs” Nat. Biotechnol. 19: 673-676.
Guatelli et al., 1990, “Isothermal, in vitro amplification of nucleic acids by a multienzyme reaction modeled after retroviral replication” Proc. Natl. Acad. Sci. 87(19): 1874-1878
Hall et al., 2000, “Sensitive detection of DNA polymorphisms by the serial invasive signal amplification reaction” Proc. Natl. Acad. Sci. USA. 97(15): 8272-8277.
Harrington et al., 1994, “The characterization of a mammalian DNA structure specific endonuclease” EMBO J. 13(5): 1235-1246
Harrington et al., 1995, “DNA structural elements required for FEN-1 binding” J. Biol. Chem. 270(9): 4503-4508.
Henricksen et al., 2000, “Inhibition of flap endonuclease 1 by flap secondary structure and relevance to repeat sequence expansion” J. Biol. Chem. 275(22): 16420-16427.
Hosfield et al., 1998, “Newly discovered archaebacterial flap endonucleases show a structure-specific mechanism for DNA substrate binding and catalysis resembling human flap endonuclease-1” J. Biol. Chem. 273(42): 27154-17161.
Hsu et al., 2001, “Genotyping single nucleotide polymorphisms by the invader assay with dual color fluorescence polarization detection” Clin. Chem. 47(8): 1373-1377.
Kaiser et al., 1999, “A comparison of eubacterial and archaeal structure-specific 5′-exonucleases” J. Biol. Chem. 274(30): 21387-21394.
Kunkel, 1992, “DNA replication fidelity” J. Biol. Chem. 267(26): 18251-18254.
Lawyer et al., 1993, “High level expression, purification, and enzymatic characterization of full-length Thermus aquaticus DNA polymerase and a truncated form deficient in 5′ to 3′ exonuclease activity” PCR Methods and Applications 2: 275-287.
Leone et al., 1998, “Molecular beacon probes combined with amplification by NABSA enable homogeneous, real-time detection of RNA” Nucleic Acids Res. 26(9): 2150-2155.
Li et al., 2002, “A new class of homogeneous nucleic acid probes based on specific displacement hybridization” Nucleic Acids Res. 30(2): e5.
Lizardi et al., 1998, “Mutation detection and single-molecule counting using isothermal rolling-circle amplification” Nature Genetics 19: 225-232.
Lundberg et al., 1991, “High-fidelity amplification using a thermostable DNA polymerase isolated from Pyrococcus furiosus” Gene 108(1): 1-6.
Lyamichev et al., 1999 “Polymorphism identification and quantitative detection of genomic DNA by invasive cleavage of oligonucleotide probes” Nat. Biotechnol. 17: 292-296.
Lyamichev et al., 1999, “Comparison of the 5′ nuclease activity of Taq DNA polymerase and its isolated nuclease domain” Proc. Natl. Acad. Sci. USA 96: 6143-6148
Lyamichev et al., 2000, “Experimental and theoretical analysis of the invasive signal amplification reaction” Biochemistry 39:9523-9532.
Ma et al., 2000, “RNA template-dependent 5′ nuclease activity of Thermus aquaticus and Thermus thermophilus DNA polymerases” J. Biol. Chem. 275(32): 24693-24700.
Matsui et al., 1999, “Thermostable flap endonuclease from the archaeon, Pyrococcus horikoshii, cleaves the replication fork-like structure endo/exonucleotically” J. Biol. Chem. 274(26): 18297-18309.
McLaughlin et al., 1984, “Chromatography purification of synthetic oligonucleotides” in Gait (editor), Oligonucleotide Synthesis—a practical approach, IRL Press, Oxford, pp117-133.
Mein et al., 2000, “Evaluation of single nucleotide polymorphism typing with invader on PCR amplicons and its automation” Genome Res. 10: 330-343.
Merkens et al., 1995, “Inactivation of the 5′-->3′ exonuclease of Thermus aquaticus DNA polymerase” Biochim. Biophys. Acta 1264: 243-248.
Murante et al., 1995, “Calf 5′ to 3′ exo/endonuclease must slide from a 5′ end of the substrate to perform structure-specific cleavage” J. Biol. Chem. 270(51): 30377-30383
Nadeau et al., 1999, “Real-time, sequence-specific detection of nucleic acids during strand displacement amplification” Anal. Biochem. 276: 177-187.
Nagamine et al., 2002, “Accelerated reaction by loop-mediated isothermal amplification using loop primers” Mol. Cell. Probes 16: 223-229.
Nilsson et al., 2002, “Real-time monitoring of rolling-circle amplification using a modified molecular beacon design” Nucleic Acids Res. 30(14): e66.
Notomi et al., 2000, “Loop-mediated isothermal amplication of DNA” Nucleic Acids Res. 28(12): e63.
Nurmi et al., 2000, “A new label technology for the detection of the detection of specific polymerase chain reaction products in a closed tube” Nucleic Acids Res. 28(8): e28
Nutiu et al., 2002, “Tripartite molecular beacons” Nucleic Acids Res. 30(18): e94. Olivier et al., 2002, “High-throughput genotyping of single nucleotide polymorphisms using new biplex invader technology” Nucleic Acids Res. 30(12): e53
Piatek et al., 1998, “Molecular beacon sequence analysis for detecting drug resistance in Mycobacterium tuberculosis” Nature Biotechnol. 16: 359-363.
Rao et al., 1998, “Methanococcus jannaschii flap endonuclease: expression, purification, and substrate requirements” J. Bacteriol. 180(20): 5406-5412.
Rumbaugh et al., 1999, “Cleavage of substrates with mismatched nucleotides by flap endonuclease-1” J. Biol. Chem. 274(21): 14602-14608
Spears et al., 1997, “Simultaneous strand displacement amplification and fluorescence polarization detection of Chlamydia trachomatis DNA” Anal. Biochem. 247: 130-137.
Tyagi et al., 1996, “Molecular Beacons: probes that fluoresce upon hybridization” Nature Biotechnol. 14: 303-308.
Tyagi et al., 1998, “Multicolor molecular beacons for allele discrimination” Nature Biotechnol. 16: 49-53.
Tyagi et al., 2000, “Wavelength-shifting molecular beacons” Nature Biotechnol. 18: 1191-1196.
Vet et al., 1998, “Multiplex detection of four pathogenic retroviruses using molecular beacons” Proc. Natl. Acad. Sci. USA. 96: 6394-6399.
Walker et al., 1992, “Isothermal in vitro amplification of DNA by a restriction enzyme/DNA polymerase system” Proc. Natl. Acad. Sci. USA 89(1): 392-396.
Walker et al., 1996, “DNA detection by strand displacement amplification and fluorescence polarization with signal enhancement using a DNA binding protein” Nucleic Acids Res. 24(2): 348-353.
Wu et al., 1984, “Purification and sequence analysis of synthetic oligodeoxyribonucleotides” in Gait (editor), Oligonucleotide Synthesis—a practical approach, IRL Press, Oxford, pp135-151.
Wu et al., 1996, “Processing of branched DNA intermediates by a complex of human FEN-1 and PCNA” Nucleic Acids Res. 24(11): 2036-2043.
Yu et al., 1997, “Specific inhibition of PCR by non-extendable oligonucleotides using a 5′ to 3′ exonuclease-deficient DNA polymerase” Biotechniques 23(4): 714-720

1. A method for generating a signal indicative of the presence of a target nucleic acid sequence in a sample comprising
incubating a sample, a probe, and a structure specific nuclease,
wherein the probe has a signaling moiety, is capable of hybridizing to a target nucleic acid sequence, and does not have a secondary structure that changes upon hybridizing of the probe to the target nucleic acid sequence,
wherein the signaling moiety is inactivated when the probe is not hybridized to the target nucleic acid sequence,
wherein the signaling moiety generates a signal by the structure specific nuclease when the probe is hybridized to the target nucleic acid sequence to form a suitable substrate for the structure specific nuclease, and
wherein the generation of the signal is indicative of the presence of a target nucleic acid sequence in the sample.

2. The method of claim 1, wherein the signaling moiety of the probe comprises
an indicating moiety and a regulating moiety separated by a site susceptible to the cleavage of the structure specific nuclease,
wherein the regulating moiety inhibits the indicating moiety when the probe is not hybridized to the target nucleic acid sequence, and wherein the structure specific nuclease separates the indicating moiety from the regulating moiety when the probe is hybridized to the target nucleic acid sequence.

3. The method of claim 2, wherein the indicating moiety is a labeled moiety and the regulating moiety is a quencher moiety which quenches the labeled moiety when the probe is not hybridized to the target nucleic acid sequence.

4. The method of claim 3, wherein the labeled moiety and the regulating moiety interacts based on a resonance energy transfer.

5. The method of claim 4, wherein the resonance energy transfer is selected from the group consisting of fluorescence resonance energy transfer, luminanscence resonance energy transfer, phosphorescence resonance energy transfer, and bioluminescence resonance energy transfer.

6. The method of claim 1, wherein the structure specific nuclease is FEN-1 or 5′ nuclease.

7. The method of claim 1, wherein the target nucleic acid sequence is a DNA sequence or an RNA sequence.

8. A method for detecting or measuring a target nucleic acid sequence in a sample comprising
incubating a sample, a probe, and a structure specific nuclease,
wherein the probe has a signaling moiety, is capable of hybridizing to a target nucleic acid sequence, and does not have a secondary structure that changes upon hybridizing of the probe to the target nucleic acid sequence,
wherein the signaling moiety is inactivated when the probe is not hybridized to the target nucleic acid sequence and the signaling moiety is activated to generate a signal by the structure specific nuclease when the probe is hybridized to the target nucleic acid sequence to form a suitable substrate for the structure specific nuclease,
detecting or measuring the amount of the signal, which is indicative of the amount of the target nucleic acid sequence present in the sample.

9. The method of claim 8, wherein the signaling moiety of the probe comprises
an indicating moiety and a regulating moiety separated by a site susceptible to the cleavage of the structure specific nuclease,
wherein the regulating moiety inhibits the indicating moiety when the probe is not hybridized to the target nucleic acid sequence, and wherein the structure specific nuclease separates the indicating moiety from the regulating moiety when the probe is hybridized to the target nucleic acid sequence.

10. The method of claim 9, wherein the indicating moiety is a labeled moiety and the regulating moiety is a quencher moiety which quenches the labeled moiety when the probe is not hybridized to the target nucleic acid sequence.

11. The method of claim 10, wherein the labeled moiety and the regulating moiety interacts based on a resonance energy transfer.

12. The method of claim 11, wherein the resonance energy transfer is selected from the group consisting of fluorescence resonance energy transfer, luminanscence resonance energy transfer, phosphorescence resonance energy transfer, and bioluminescence resonance energy transfer.

13. The method of claim 8, wherein the structure specific nuclease is FEN-1 or 5′ nuclease.

14. The method of claim 8, wherein the target nucleic acid sequence is a DNA sequence or an RNA sequence.

15. A method for generating a signal indicative of the presence of a target nucleic acid sequence in a sample comprising
incubating a sample, a first probe, and a structure specific nuclease,
amplifying a sequence containing a target nucleic acid sequence in the sample including using a nucleic acid polymerase which does not have any substantial 5′-3′ nuclease activity
wherein the first probe has a signaling moiety and is capable of hybridizing to the target nucleic acid sequence,
wherein the signaling moiety is inactivated when the first probe is not hybridized to the target nucleic acid sequence,
wherein the signaling moiety generates a signal by the structure specific nuclease when the first probe is hybridized to the target nucleic acid sequence to form a suitable substrate for the structure specific nuclease, and
wherein the generation of the signal is indicative of the presence of a target nucleic acid sequence in the sample.

16. The method of claim 15, wherein the suitable substrate for the structure specific nuclease includes a double-strand duplex, a double-strand duplex with a 5′ flap, or a double-strand duplex with a 3′ flap.

17. The method of claim 15 further comprises incubating a second probe capable of hybridizing to a region 5′ upstream of the target nucleic acid sequence.

18. The method of claim 17 wherein the second probe is capable of hybridizing to a region 5′ upstream and juxtaposed to the target nucleic acid sequence.

19. The method of claim 17 wherein the second probe is capable of hybridizing to a region 5′ upstream and overlapping with the target nucleic acid sequence.

20. The method of claim 15, wherein the first probe hybridizes to the target nucleic acid sequence with a 3′ flap or a 5′ flap.

21. The method of claim 15, wherein the first probe contains a 3′ end which is not extendable by the nucleic acid polymerase.

22. The method of claim 17, wherein the second probe is a primer used for amplifying the sequence containing the target nucleic acid sequence.

23. The method of claim 17, wherein the second probe hybridizes to the 5′ upstream region of the target nucleic acid sequence with a 5′ flap or a 3′ flap.

24. The method of claim 15, wherein the signaling moiety of the probe comprises
an indicating moiety and a regulating moiety separated by a site susceptible to the cleavage of the structure specific nuclease,
wherein the regulating moiety inhibits the indicating moiety when the probe is not hybridized to the target nucleic acid sequence, and wherein the structure specific nuclease separates the indicating moiety from the regulating moiety when the probe is hybridized to the target nucleic acid sequence.

25. The method of claim 24, wherein the indicating moiety is a labeled moiety and the regulating moiety is a quencher moiety which quenches the labeled moiety when the probe is not hybridized to the target nucleic acid sequence.

26. The method of claim 25, wherein the labeled moiety and the regulating moiety interacts based on a resonance energy transfer.

27. The method of claim 26, wherein the resonance energy transfer is selected from the group consisting of fluorescence resonance energy transfer, luminanscence resonance energy transfer, phosphorescence resonance energy transfer, and bioluminescence resonance energy transfer.

28. The method of claim 15, wherein the structure specific nuclease is FEN-1 or 5′ nuclease.

29. The method of claim 15, wherein the target nucleic acid sequence is a DNA sequence or an RNA sequence.

30. The method of claim 15, wherein the sequence containing the target nucleic acid sequence is amplified by isothermal amplification, thermocycling amplification, linear amplification, or exponential amplification.

31. A method for detecting or measuring the presence of a target nucleic acid sequence in a sample comprising
incubating a sample, a first probe, and a structure specific nuclease,
amplifying a sequence containing a target nucleic acid sequence in the sample including using a nucleic acid polymerase which does not have any substantial 5′-3′ nuclease activity
wherein the first probe has a signaling moiety and is capable of hybridizing to the target nucleic acid sequence,
wherein the signaling moiety is inactivated when the first probe is not hybridized to the target nucleic acid sequence, and
wherein the signaling moiety generates a signal by the structure specific nuclease when the first probe is hybridized to the target nucleic acid sequence to form a suitable substrate for the structure specific nuclease,
detecting or measuring the amount of signal, which is indicative of the amount of the target nucleic acid sequence present in the sample.

32. The method of claim 31, wherein the suitable substrate for the structure specific nuclease includes a double-strand duplex, a double-strand duplex with a 5′ flap, or a double-strand duplex with a 3′ flap.

33. The method of claim 31 further comprises incubating a second probe capable of hybridizing to a region 5′ upstream of the target nucleic acid sequence.

34. The method of claim 33 wherein the second probe is capable of hybridizing to a region 5′ upstream and juxtaposed to the target nucleic acid sequence.

35. The method of claim 33 wherein the second probe is capable of hybridizing to a region 5′ upstream and overlapping with the target nucleic acid sequence.

36. The method of claim 31, wherein the first probe hybridizes to the target nucleic acid sequence with a 3′ flap or a 5′ flap.

37. The method of claim 31, wherein the first probe contains a 3′ end which is not extendable by the nucleic acid polymerase.

38. The method of claim 33, wherein the second probe is a primer used for amplifying the sequence containing the target nucleic acid sequence.

39. The method of claim 33, wherein the second probe hybridizes to the 5′ upstream region of the target nucleic acid sequence with a 5′ flap or a 3′ flap.

40. The method of claim 31, wherein the signaling moiety of the probe comprises
an indicating moiety and a regulating moiety separated by a site susceptible to the cleavage of the structure specific nuclease,
wherein the regulating moiety inhibits the indicating moiety when the probe is not hybridized to the target nucleic acid sequence, and wherein the structure specific nuclease separates the indicating moiety from the regulating moiety when the probe is hybridized to the target nucleic acid sequence.

41. The method of claim 40, wherein the indicating moiety is a labeled moiety and the regulating moiety is a quencher moiety which quenches the labeled moiety when the probe is not hybridized to the target nucleic acid sequence.

42. The method of claim 41, wherein the labeled moiety and the regulating moiety interacts based on a resonance energy transfer.

43. The method of claim 42, wherein the resonance energy transfer is selected from the group consisting of fluorescence resonance energy transfer, luminanscence resonance energy transfer, phosphorescence resonance energy transfer, and bioluminescence resonance energy transfer.

44. The method of claim 31, wherein the structure specific nuclease is FEN-1 or 5′ nuclease.

45. The method of claim 31, wherein the target nucleic acid sequence is a DNA sequence or an RNA sequence.

46. The method of claim 31, wherein the sequence containing the target nucleic acid sequence is amplified by isothermal amplification, thermocycling amplification, linear amplification, or exponential amplification.

47. A kit comprising a first probe and a structure specific nuclease,
wherein the first probe has a signaling moiety, is capable of hybridizing to a target nucleic acid sequence, and does not have a secondary structure that changes upon hybridizing of the first probe to the target nucleic acid sequence, and
wherein the signaling moiety is inactivated when the first probe is not hybridized to the target nucleic acid sequence and the signaling moiety is activated to generate a signal by the structure specific nuclease when the first probe is hybridized to the target nucleic acid sequence to form a suitable substrate for the structure specific nuclease.

48. The kit of claim 47, wherein the signaling moiety of the first probe comprises
an indicating moiety and a regulating moiety separated by a site susceptible to the cleavage of the structure specific nuclease,
wherein the regulating moiety inhibits the indicating moiety when the first probe is not hybridized to the target nucleic acid sequence, and wherein the structure specific nuclease separates the indicating moiety from the regulating moiety when the first probe is hybridized to the target nucleic acid sequence.

49. The kit of claim 48, wherein the indicating moiety is a labeled moiety and the regulating moiety is a quencher moiety which quenches the labeled moiety when the first probe is not hybridized to the target nucleic acid sequence.

50. The kit of claim 49, wherein the labeled moiety and the regulating moiety interacts based on a resonance energy transfer.

51. The kit of claim 50, wherein the resonance energy transfer is selected from the group consisting of fluorescence resonance energy transfer, luminanscence resonance energy transfer, phosphorescence resonance energy transfer, and bioluminescence resonance energy transfer.

52. The kit of claim 47, wherein the structure specific nuclease is FEN-1 or 5′ nuclease.

53. The kit of claim 47, wherein the target nucleic acid sequence is a DNA sequence or an RNA sequence.

54. A kit comprising a first probe, a structure specific nuclease, and a nucleic acid polymerase which does not have any substantial 5′-3′ nuclease activity,
wherein the first probe has a signaling moiety and is capable of hybridizing to a target nucleic acid sequence,
wherein the nucleic acid polymerase is capable of amplifying a sequence containing the target nucleic acid sequence,
wherein the signaling moiety is inactivated when the first probe is not hybridized to the target nucleic acid sequence, and
wherein the signaling moiety generates a signal by the structure specific nuclease when the first probe is hybridized to the target nucleic acid sequence to form a suitable substrate for the structure specific nuclease.

55. The kit of claim 54, wherein the suitable substrate for the structure specific nuclease includes a double-strand duplex, a double-strand duplex with a 5′ flap, or a double-strand duplex with a 3′ flap.

56. The kit of claim 54 further comprises a second probe capable of hybridizing to a region 5′ upstream of the target nucleic acid sequence.

57. The kit of claim 56 wherein the second probe is capable of hybridizing to a region 5′ upstream and juxtaposed to the target nucleic acid sequence.

58. The kit of claim 56 wherein the second probe is capable of hybridizing to a region 5′ upstream and overlapping with the target nucleic acid sequence.

59. The kit of claim 54, wherein the first probe is capable of hybridizing to the target nucleic acid sequence with a 3′ flap or a 5′ flap.

60. The kit of claim 54, wherein the first probe contains a 3′ end which is not extendable by the nucleic acid polymerase.

61. The kit of claim 56, wherein the second probe is a primer used for amplifying the sequence containing the target nucleic acid sequence.

62. The kit of claim 56, wherein the second probe is capable of hybridizing to the 5′ upstream region of the target nucleic acid sequence with a 5′ flap or a 3′ flap.

63. The kit of claim 54, wherein the signaling moiety of the probe comprises
an indicating moiety and a regulating moiety separated by a site susceptible to the cleavage of the structure specific nuclease,
wherein the regulating moiety inhibits the indicating moiety when the probe is not hybridized to the target nucleic acid sequence, and wherein the structure specific nuclease separates the indicating moiety from the regulating moiety when the probe is hybridized to the target nucleic acid sequence.

64. The kit of claim 63, wherein the indicating moiety is a labeled moiety and the regulating moiety is a quencher moiety which quenches the labeled moiety when the probe is not hybridized to the target nucleic acid sequence.

65. The kit of claim 64, wherein the labeled moiety and the regulating moiety interacts based on a resonance energy transfer.

66. The kit of claim 65, wherein the resonance energy transfer is selected from the group consisting of fluorescence resonance energy transfer, luminanscence resonance energy transfer, phosphorescence resonance energy transfer, and bioluminescence resonance energy transfer.

67. The kit of claim 54, wherein the structure specific nuclease is FEN-1 or 5′ nuclease.

68. The kit of claim 54, wherein the target nucleic acid sequence is a DNA sequence or an RNA sequence.

69. The kit of claim 54, wherein the nucleic acid polymerase and the structure specific nuclease is a recombinant protein.

70. The kit of claim 54, wherein the nucleic acid polymerase and the structure specific nuclease is provided as a single recombinant protein.

71. The kit of claim 54 further comprises at least one amplification primer useful for amplifying the sequence containing the target nucleic acid sequence.

72. The kit of claim 54 further comprises a forward amplification primer and a reverse amplification primer useful for amplifying the sequence containing the target nucleic acid sequence.
